(19) United States
(12) Patent Application Publication
(10) Pub. No.: US 2024/0261445 A1
(43) Pub. Date: Aug. 8, 2024

(54) PRECISION RADIOPHARMACEUTICAL THERAPY OF LYMPHOMA AND OTHER DISEASES

(71) Applicants: Richard Wahl, St. Louis, MO (US); Mark Longtine, St. Louis, MO (US); Kyu-Hwan Shim, St. Louis, MO (US); Mark Hoegger, St. Louis, MO (US)

(72) Inventors: Richard Wahl, St. Louis, MO (US); Mark Longtine, St. Louis, MO (US); Kyu-Hwan Shim, St. Louis, MO (US); Mark Hoegger, St. Louis, MO (US)

(73) Assignee: Washington University, St. Louis, MO (US)

(21) Appl. No.: 18/434,796

(22) Filed: Feb. 6, 2024

Related U.S. Application Data
(60) Provisional application No. 63/483,532, filed on Feb. 6, 2023.

(51) Int. Cl. A61K 51/10; A61P 35/00

(52) U.S. Cl. CPC A61K 51/1096; A61K 51/1027; A61P 35/00; A61K 2121/00; A61K 2123/00

(57) ABSTRACT

The disclosure allows precision dosing of the therapeutic antibodies to individual patients based on their own, imaged, pharmacokinetics.

### CROSS-REFERENCE TO RELATED APPLICATIONS

This application claims priority from U.S. Provisional Application Ser. No. 63/483,532 filed on 6 Feb. 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to the biomedical field, particularly to the methods of administering therapeutic antibodies which provide better clinical outcomes.

### BRIEF DESCRIPTION OF THE DISCLOSURE

In one aspect of the present disclosure, a method of treating disseminated human lymphoma in a subject in need thereof is provided. The method comprises applying a theranostic treatment comprising at least one of [²²⁵Ac]Ac-Ofatumumab and [177Lu]Lu-ofatumumab.

In another aspect of the present disclosure, a method of treating disseminated human lymphoma in a subject in need thereof is provided. The method comprises applying a theranostic treatment comprising a combination of [²²⁵Ac]Ac-Ofatumumab and [89Zr]Zr-Ofatumumab.

In a further aspect of the present disclosure, a method of treating a CD20 disease in a subject in need thereof is provided. The method comprises applying a theranostic treatment comprising at least one of [²²⁵Ac]Ac-Ofatumumab, [177Lu]Lu-ofatumumab, and a combination of [²²⁵Ac]Ac-Ofatumumab and [89Zr]Zr-Ofatumumab.

### DESCRIPTION OF THE DRAWINGS

Those of skill in the art will understand that the drawings disclosed herein are for illustrative purposes only. The drawings are not intended to limit the scope of the present teachings in any way.

FIG. 1(A-H) is an exemplary embodiment of quality control characterization and biodistribution of [225Ac]AcDOTA-ofatumumab in accordance with the present disclosure. FIG. 1A: Chemical structure. FIG. 1B and FIG. 1C: Radiochemical purity by TLC (FIG. 1B) or FPLC (FIG. 1C) showing UV absorbance (solid line) and γ counting of fractions at secular equilibrium as counts per minute (cpm, dotted line). Antibody elutes at 13 min and DTPA, detected by UV, elutes at 19 min. n=7. FIG. 1D: Radiochemical yields. n=7. FIG. 1E: Immunoreactivity assayed by Rajicell binding. n=3. FIG. 1F: Average DOTA molecules per ofatumumab. FIG. 1G: Stability in human serum at 37° C. n=3. FIG. 1H: Biodistribution in R2G2 mice with subcutaneous Raji tumors 7-d after injection of [225Ac]Ac-ofatumumab (n=4). ***, P<0.001.

FIG. 2 is an exemplary embodiment of [²²⁵Ac]Ac-ofatumumab in vitro cytotoxicity to Raji-Luc cells in accordance with the present disclosure. Cell viability with no treatment control or after exposure to [²²⁵Ac]Ac-IgG, or -ofatumumab was measured using MTS (left) or bioluminescence (right) assays (n=3). Comparison was by ANOVA (red lines) or Student t test (black and blue lines). *P<0.05. **P<0.005. Ab=antibody; Ofa=ofatumumab.

FIG. 3(A-B) is an exemplary embodiment of survival and bioluminescence of mice with disseminated Raji-Luc cells untreated or receiving therapy 8 d after cell injection in accordance with the present disclosure. FIG. 3A: Kaplan-Meier graph with median survivals. Low-dose [²²⁵Ac]Ac-ofatumumab was superior to all other cohorts except high-dose ofatumumab (Mantel-Cox, P<0.05). High-dose [²²⁵Ac]Ac-ofatumumab was superior to all other cohorts. FIG. 3B: Bioluminescence. Note elimination of Raji-Luc cells in 9.25 kBq/mouse cohort. Arrow indicates initiation of therapy. n=10/cohort. BLI=bioluminescence.

FIG. 4 is an exemplary embodiment of bioluminescence of mice with disseminated Raji-Luc cells untreated or receiving therapy 8 d after cell injection in accordance with the present disclosure. Images shown were obtained just before censoring or study termination. Median days of imaging were 20 d for no treatment and ofatumumab; 20 and 23 d for low- and high-dose [²²⁵Ac]Ac-IgG, respectively; and 152 and 198 d for low- and high-dose [²²⁵Ac]Ac-ofatumumab, respectively. Note elimination of bioluminescence in mice in latter 2 cohorts. Censoring was for HLP, weight loss, and other causes. C=other causes; SUR=survival; WL=weight loss.

FIG. 5(A-B) is an exemplary embodiment of rapidity of therapeutic effects in mice treated 8 d after cell injection in accordance with the present disclosure. FIG. 5A: Bioluminescence of mice 3-20 d after cell injection, showing profound reduction of cancer cell growth rates by [²²⁵Ac]Ac-ofatumumab. FIG. 5B: Comparison by ANOVA of bioluminescence from 2 to 10 d after therapy initiation (10-20 d after cell injection). n=7-10/cohort. BLI=bioluminescence; ns=not statistically significant. **P<0.005.

FIG. 6(A-B) is an exemplary embodiment of weights of control naïve mice, and in untreated or treated mice with therapy initiated 8-d after cell injection in accordance with the present disclosure. FIG. 6A: Weights of mice injected intravenously with Rajiluc cells and 8-d later left untreated or treated. Note, the two surviving mice in the no treatment and high-dose [²²⁵Ac]Ac-IgG cohorts likely had unsuccessful injections/engraftments. FIG. 6B: Weights of control naïve (not injected with cells, no therapy) mice and of mice treated with lower high-dose [225Ac]Ac-ofatumumab. Right: Comparison by ANOVA. *, P<0.05. **, P<0.005. Naïve mice, n=4-5. 12-d therapy, n=9. 16-d therapy, n=8.

FIG. 7 is an exemplary embodiment of bioluminescence of untreated, Raji-luc injected mice at initial imaging and 8-, 12- or 16-d after cell injection in accordance with the present disclosure. Initial imaging, n=73, 8-, 12- and 16-d after cell injection, n=25, 25 and 33, respectively. BLI values were normalized to the first imaging day and log-transformed values were compared by ANOVA. Values are shown as average±standard deviation, with 8-, 12- and 16-d showing log-fold increases increases of 1.04±0.26, 1.41±0.20 and 1.67±0.25, respectively. ***, all comparisons showed P<0.001.

FIG. 8(A-C) is an exemplary embodiment of survival and bioluminescence of mice with larger pretreatment cancer-cell burden in accordance with the present disclosure. FIG. 8A: Kaplan-Meier graph of untreated mice vs. treatment started 16 d (left) or 12 d (right) after cell injection, with median survival indicated. Significance was evaluated by Mantel-Cox test. FIG. 8B: Bioluminescence, showing effect on cancer-cell numbers for cohort treated on day 12 but not day 16. FIG. 8C: ANOVA comparison of cancer-cell growth of untreated mice or mice treated 12 d after cell injection. n=7-9/cohort. BLI=bioluminescence; ns=not statistically significant. ***P<0.0001.

FIG. 9(A-C) is an exemplary embodiment of bioluminescence images over the life span of mice with disseminated Raji-luc cells in accordance with the present disclosure. FIG. 9A: left untreated. FIG. 9B, FIG. 9C, and FIG. 9D: treated with 9.25 kBq of [²²⁵Ac]Ac-ofatumumab on 16-d (FIG. 9B) or 12-d (FIG. 9C: 2 through 27 days, and FIG. 9D: 27 through 40 days) after cell injection. n=8-9/cohort HLP, sacrifice for hind-limb paralysis. WL, sacrifice for >20% loss of initial weight. P, perished.

FIG. 10(A-C) is an exemplary embodiment of [177Lu]Lu-ofatumumab in vitro stability, cell killing and affinity in accordance with the present disclosure. FIG. 10A: Stability in serum at 37° C. and buffer at 4° C. (n=3). FIG. 10B: Killing of Raji-luc cells (n=3). Student's unpaired t-test, comparing [177Lu]Lu-ofatumumab and [¹⁷⁷Lu]Lu-IgG. *, p<0.05, ***, p<0.0001. FIG. 10C: Raji-cell saturation binding assay shown at two scales, with or without blocking (n=3). Cpm, counts per minute. Ofa, ofatumumab.

FIG. 11 is an exemplary embodiment of weights of mice with disseminated Raji-luc cells with therapy initiated 4 d post cell injection in accordance with the present disclosure. ANOVA tests of 8.51 MBq [¹⁷⁷Lu]Lu-ofatumumab treated mice, comparing to d 0 weights, indicated weight loss from 10 d to 35 d and weight gain after 75 d (p<0.05). n=10 mice/group. Ofa, ofatumumab.

FIG. 12 is an exemplary embodiment of tumor-cell growth in mice one to eighteen d post initiation of therapy in accordance with the present disclosure. Radiance (photons/sec/cm²/steradian) values of individual mice and the mean±standard deviation of mice treated with 8.51 MBq of [¹⁷⁷Lu]Lu-ofatumumab are shown. Ofa, ofatumumab.

FIG. 13 is an exemplary embodiment of [¹⁷⁷Lu]Lu-ofatumumab biodistribution in R2G2 mice with subcutaneous Raji tumors in accordance with the present disclosure. Biodistribution was assayed 1, 3, or 7 d after radiopharmaceutical injection (3-16 mice per time point), with data presented as mean±SD. One-way ANOVA compares distribution in organ or tissue at each time point. *P<0.05. **P<0.001. ***P<0.0001. Sm Int=small intestine; U Lg Int=upper large intestine; L Lg Int=lower large intestine.

FIG. 14 is an exemplary embodiment of survival analysis of mice with disseminated Raji-luc cells with therapy initiated 4 d after cell injection in accordance with the present disclosure. Kaplan-Meier graph shows median survival, in days. Ofa=ofatumumab.

FIG. 15(A-B) is an exemplary embodiment of tumor burden of mice with disseminated Raji-luc cells with therapy initiated 4 d after cell injection in accordance with the present disclosure. FIG. 15A: Bioluminescence (10 mice per group). FIG. 15B: Representative bioluminescence images at indicated days after cell injection. Radiance is photons/s/cm²/steradian. Ofa=ofatumumab.

FIG. 16 is an exemplary embodiment of bioluminescence images of untreated or treated mice with disseminated Raji-luc cells on final imaging event before mice were killed for cause or study termination in accordance with the present disclosure. Ofa=ofatumumab.

FIG. 17 is an exemplary embodiment of tumor-cell growth in mice 1-18 d after initiation of therapy in accordance with the present disclosure. Log of slopes of radiance (photons/s/cm²/steradian) over this time are shown as mean±SD and were analyzed by ANOVA, comparing all samples with each other (10 mice per group). *P<0.05. **P<0.005. ***P<0.0001. Ofa=ofatumumab.

### DETAILED DESCRIPTION

Ac225 anti CD20 is a highly active cancer therapeutic and has been shown to cure lymphoma in mice if tumor burden is low. Lu177 anti CD20 can cure lymphoma, though best suited to high tumor burden situations. Zr89 anti CD20 images lymphoma well. Disclosed herein is Ac225 anti CD20 as a cancer therapeutic with doses and sequences defined by Zr89 anti CD20, as well as Lu177 anti CD20 imaging. While Ac 225 can only be imaged poorly, the present disclosure contemplates situations in which patients are imaged with low count anti CD20 images/quantitation, including a combination of Ac225 and Zr89.

In one aspect of the present disclosure, Ac225 anti CD20 is utilized as a cancer therapeutic with Zr89 anti cD20 to predict Ac225 anti CD20 biodistribution. In another aspect of the present disclosure, Ac225 anti CD20 is utilized as a cancer therapeutic with Lu177 anti cD20 to predict Ac225 anti CD20 biodistribution. In yet another aspect of the present disclosure, Ac225 anti CD20 is utilized as a cancer therapeutic with both Zr89 and Lu177 anti cD20 to predict Ac225 anti CD20 biodistribution. In some embodiments, treatment of non-malignant disease is conducted with a sequential imaging and dosing method.

In the present disclosure, a precision radiopharmaceutical therapy of NHL is developed which includes Zr89 ofatumomab (for imaging and predicting dosimetry), Lu177 ofatumomab for therapy and dosimetry, and ultimately Ac225 ofatumomab for the optimal treatment of low tumor burden foci. Aim 1 explores both disseminated and solid tumor lymphoma models (as well as the combination of both disease sites model) the efficacy of Lu 177 ofatumomab and Ac225 ofatumomab as monotherapies, either as single dose or multiple dose regimens, including combined and sequential dosing to determine which regimen is most curative. Aim 1 accordingly informs Aim 2, which is a first in man study of Zr89 ofatumomab for imaging and predicted tumor dosimetry. Aim 2 is conducted in patients with recurrent diffuse large B cell lymphoma and includes “no rituximab pre-dose” and rituximab pre-dose groups, to determine if and how pre-dosing with unlabeled antibody changes/improves radiation absorbed dose. In Aim 3, patients from Aim 2 receive a therapy dose of Lu177 ofatumomab in a standard phase I dose escalation trial with dose escalation based on predicted bone marrow radiation absorbed dose. These therapy doses are followed by post treatment dosimetry images to measure actual radiation absorbed dose to tumors (from Lu177) vs predicted dose from Zr89 PET. These aims and studies inform subsequent Ac225 ofatumomab clinical studies once stable Ac225 supplies for human use are available. Overall, the disclosed regimens have highly curative potential of otherwise incurable NHL.

The disclosed regimens can be varied with differing imaging, beta therapy and alpha therapy emitters given sequentially or in combination. The disclosed regimens are applicable to malignant and benign diseases with a variety of targets, and are shown using the CD20 targeting space. The disclosed approach has broad utility to CD20 positive non-malignant diseases as well as to other targets.

As described herein, Non-Hodgkin Lymphoma (NHL) is curable with the disclosed approach. Using patient specific dosimetry and sequencing of therapy, precise patient specific radiation dosing can be delivered. Further, the anti CD20 radiolabeled antibody labeled with Ac225 and developed herein has broad applicability in treating autoimmune diseases. Further, the sequencing approach described herein also has broad applicability (e.g. imaging/high beta energy therapy/alpha emitter therapy).

In some aspects of the present disclosure, Ac225 therapy (alone or in combination with Zr89 is utilized as a cancer therapeutic for NHL as well as other CD20 positive diseases, including multiple sclerosis (MS). In some embodiments, administration of treatment therapy is sequential with precision imaging, beta and alpha emitters, given sequentially or simultaneously.

Immunotherapies that target the CD20 protein expressed on most non-Hodgkin lymphoma cells have improved clinical outcomes, but relapse is common. ²²⁵Ac-labeled anti-CD20 ofatumumab was prepared and evaluated for in vitro characteristics and therapeutic efficacy in a murine model of disseminated human lymphoma.

²²⁵Ac was chelated by DOTA-ofatumumab, and radiochemical yield, purity, immunoreactivity, stability, and chelate number were determined. In vitro cell killing of CD20-positive, human B-cell lymphoma Raji-Luc cells was assayed. Biodistribution was determined as percentage injected activity per gram (% IA/g) in mice with subcutaneous Raji-cell tumors (n=4). [²²⁵Ac]Ac-ofatumumab biodistribution in C57BL/6N mice was performed to estimate projected human dosimetry. Therapeutic efficacy was tested in mice with systemically disseminated Raji-Luc cells, tracking survival, bioluminescence, and animal weight for a targeted 200 d, with single-dose therapy initiated 8, 12, or 16 d after cell injection, comparing no treatment, ofatumumab, and low (3.7 kBq/mouse) and high (9.25 kBq/mouse) doses of [²²⁵Ac]Ac-IgG and [²²⁵Ac]Ac-ofatumumab (n=8-10/cohort).

Radiochemical yield and purity were 32%±9% and more than 95%, respectively. Specific activity was more than 5 MBq/mg. Immunoreactivity was preserved, and more than 90% of the ²²⁵Ac remained chelated after 10 d in serum. Raji-Luc cell killing in vitro was significant, specific, and dose-dependent. In tumor-bearing mice, [²²⁵Ac]Ac-ofatumumab displayed low liver (7% IA/g) and high tumor (28% IA/g) uptake. Dosimetry estimates indicated that bone marrow is likely the dose-limiting organ. When therapy was initiated 8 d after cell injection, untreated mice and mice treated with cold ofatumumab or low- or high-dose [²²⁵Ac]Ac-IgG showed indistinguishable median survivals of 20-24 d, with extensive cancer-cell burden before death. Low- and high-dose [²²⁵Ac]Ac-ofatumumab profoundly (P<0.05) extended median survival to 190 d and more than 200 d (median not determinable), with 5 and 9 of 10 mice, respectively, surviving at study termination with no detectable cancer cells. Surviving mice treated with high-dose [²²⁵Ac]Ac-ofatumumab showed reduced weight gain versus naïve mice. When therapy was initiated 12 d, but not 16 d, after cell injection, high-dose [²²⁵Ac]Ac-ofatumumab significantly extended median survival to 40 d but was not curative.

In an aggressive disseminated tumor model, [²²⁵Ac]Ac-ofatumumab was effective at cancer-cell killing and curative when administered 8 d after cell injection. [²²⁵Ac]Ac-ofatumumab has substantial potential for clinical translation as a next-generation therapeutic for treatment of patients with non-Hodgkin lymphoma.

Although immunotherapies that target CD20 on most non-Hodgkin lymphoma (NHL) cells have improved patient outcomes, current therapies are inadequate because many cases are, or become, refractory or undergo relapse. Here, the third-generation human anti-CD20 antibody ofatumumab was labelled with 177Lu, determined the in vitro characteristics of [177Lu]Lu-ofatumumab, estimated human dosimetry, and assayed tumor targeting and therapeutic efficacy in a murine model of disseminated NHL.

CHX-A99-diethylenetriaminepentaacetic acid-[¹⁷⁷Lu]Lu-ofatumumab was prepared. Radiochemical yield, purity, in vitro immunoreactivity, stability, (n 5 7), affinity, and killing of CD20-expressing Raji cells (n5 3) were evaluated. Human dosimetry was estimated from biodistribution studies as percentage injected activity per gram using C57BL/6N mice. Tissue and organ biodistribution was determined in R2G2 immunodeficient mice with subcutaneous Raji-cell tumors. Therapy studies used R2G2 mice with disseminated human Raji-luc tumor cells (n 5 10 mice/group). Four days after cell injection, the mice were left untreated or were treated with ofatumumab, 8.51 MBq of [¹⁷⁷Lu]Lu-IgG, or 0.74 or 8.51 MBq of [¹⁷⁷Lu]Lu-ofatumumab. Survival, weight, and bioluminescence were tracked.

Radiochemical yield was 93% 6 2%, radiochemical purity was 99% 6 1%, and specific activity was 401 6 17 MBq/mg. Immunoreactivity was substantially preserved, and more than 75% of ¹⁷⁷Lu remained chelated after 7 d in serum. [¹⁷⁷Lu]Lu-ofatumumab specifically killed Rajiluc cells in vitro (P, 0.05). Dosimetry estimated that an effective dose for human administration is 0.36 mSv/MBq and that marrow may be the dose-limiting organ. Biodistribution in subcutaneous tumors 1, 3, and 7 d after [¹⁷⁷Lu]Lu-ofatumumab injection was 11, 15, and 14 percentage injected activity per gram, respectively. In the therapy study, median survival of untreated mice was 19 d, not statistically different from mice treated with 8.51 MBq of [¹⁷⁷Lu]Lu-IgG (25 d). Unlabeled ofatumumab increased survival to 46 d, similar to 0.74 MBq of [¹⁷⁷Lu]Lu-ofatumumab (59 d), with both being superior to no treatment (P, 0.0003). Weight loss and increased tumor burden preceded death or killing of the animal for cause. In contrast, treatment with 8.51 MBq of [¹⁷⁷Lu]Lu-ofatumumab dramatically increased median survival (0.221 d), permitted weight gain, eliminated detectable tumors, and was curative in 9 of 10 mice.

[¹⁷⁷Lu]Lu-ofatumumab shows favorable in vitro characteristics, localizes to tumor, and demonstrates curative therapeutic efficacy in a disseminated lymphoma model, showing potential for clinical translation to treat NHL.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° ° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Methods described herein are generally performed on a subject in need thereof. A subject in need of the therapeutic methods described herein can be a subject having, diagnosed with, suspected of having, or at risk for developing disseminated human lymphoma or non-Hodgkin's lymphoma. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a theranostic treatment agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to cure disseminated human lymphoma or non-Hodgkin's lymphoma.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as in some embodiments the necessary therapeutically effective amount is reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of theranostic treatment agents can occur as a single event or over a time course of treatment. For example, theranostic treatment agents can be administered daily, weekly, bi-weekly, or monthly. For treatment of acute conditions, the time course of treatment will usually be at least several days. Depending upon the embodiment, certain conditions extend treatment from several days to several weeks. For example, in some embodiments treatment extends over one week, two weeks, or three weeks. For more chronic conditions, treatment extends from several weeks to several months or even a year or more.

Treatment in accord with the methods described herein can be performed prior to or before, concurrent with, or after conventional treatment modalities for disseminated human lymphoma.

A theranostic treatment agent can be administered simultaneously or sequentially with another agent, such as an antibiotic, an anti-inflammatory, or another agent. For example, a theranostic treatment agent can be administered simultaneously with another agent, such as an antibiotic or an anti-inflammatory. Simultaneous administration can occur through administration of separate compositions, each containing one or more of a theranostic treatment agent, an antibiotic, an anti-inflammatory, or another agent. Simultaneous administration can occur through administration of one composition containing two or more of a theranostic treatment agent an antibiotic, an anti-inflammatory, or another agent. A theranostic treatment agent can be administered sequentially with an antibiotic, an anti-inflammatory, or another agent. For example, a theranostic treatment agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the theranostic treatment agents may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, theranostic treatment agents may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. Some embodiments involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods. The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to theranostic treatment agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the found to function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Cure of Disseminated Human Lymphoma with [²²⁵Ac]Ac-Ofatumumab in a Preclinical Model

This Example describes a novel therapeutic approach for treating cancer and other diseases, including lymphoma and MS, with [²²⁵Ac]Ac-Ofatumumab alone or in combination with [⁸⁹Zr]Zr-Ofatumumab.

Non-Hodgkin lymphoma (NHL) will be diagnosed in approximately 80,000 patients and cause over 20,000 deaths in the United States in 2023. Although chemotherapy is initially effective, many patients, even with low-grade lymphomas, relapse. This has driven development of therapeutic antibodies that target the CD20 protein expressed on the surface of mature B cells and most NHL cells, as most are of B-cell origin. Anti-CD20 immunotherapy has a highly favorable safety profile, significantly improves the outcomes of most patients, and, along with chemotherapy, is now part of the standard of care for many cases of NHL.

The chimeric mouse-human monoclonal antibody, rituximab, was the first Food and Drug Administration-approved anti-CD20 therapeutic, with others subsequently developed for improved biologic and pharmacologic properties, including fully human ofatumumab. Ofatumumab binds CD20 with high affinity, allowing targeting of cells with low CD20 expression, including those with resistance to rituximab. As a type I anti-CD20 antibody, ofatumumab is effectively internalized, which benefits imaging and therapy using residualizing radiometals, such as ⁸⁹Zr and ²²⁵Ac.

Lymphoma is highly susceptible to ionizing radiation; however, external-beam irradiation is used sparingly in the disseminated setting. To overcome the limitations of external-beam radiotherapy, systemically administered β-particle-emitting radioimmunotherapies to anti-CD20 have been translated into 2 Food and Drug Administration-approved drugs: murine ¹³¹I-tositumomab (Bexxar; GlaxoSmithKline) and ⁹⁰Y-ibrutumomab (Zevalin; Acrotech Biopharma LLC). Although studies show long-term safety and effectiveness, Bexxar has been discontinued commercially in the United States, and Zevalin is used infrequently.

²²⁵Ac has gained increased use as a therapeutic radionuclide, with its 10-d half-life matching well the pharmacokinetics of intact antibodies. In its decay pathway, ²²⁵Ac yields 4 net α-particles with high linear energy transfer and short pathlengths, providing effective radiotoxicity to targeted tumor cells while relatively sparing nontargeted cells.

It has been demonstrated that [⁸⁹Zr]Zr-ofatumumab has excellent uptake into human lymphoma xenografts and enables in vivo localization using PET as well as, or better than, [⁸⁹Zr]Zr-rituximab. As disclosed herein, [²²⁵Ac]Ac-ofatumumab was prepared and demonstrated to have potent cytotoxicity to CD20-expressing cells in vitro and in vivo. Exemplary embodiments of the present disclosure include [²²⁵Ac]Ac-ofatumumab and combination [²²⁵Ac]Ac-ofatumumab+[⁸⁹Zr]Zr-ofatumumab treatments for cancer and other CD20 diseases such as MS In therapeutic studies using an aggressive, disseminated murine model of human lymphoma, [²²⁵Ac]Ac-ofatumumab shows excellent, often curative, efficacy.

### Materials and Methods

### Reagents and Cell Lines

Chemicals and reagents are listed in TABLE 2. Water (MilQ Integral 5 system; Millipore) was treated with a 50 g/L concentration of Chelex 100 (Bio-Rad Laboratories, Inc.). Raji and Raji-Luc cells were cultured in RPMI medium with 10% fetal bovine serum.

TABLE 2
Reagents/Supplies.

| Item | Company | Catalog number | Description/Use |
|---|---|---|---|
| Ofatumumab | Novartis | | IgG1κ anti-CD20 antibody |
| IgG1κ | BioXcel | BE0297 | Control antibody |
| Actinium-225 | Oak | 0010 | Radioisotope |
| (generator | Ridge | | |
| produced) | National | | |
| | Laboratory | | |
| Raji cells | ATCC | CCL-86 | Cell line |
| Raji-luc cells | Irving | Gift | Cell line |
| | Weissman | | |
| RPMI | Corning | MT10040CV | Cell culture medium |
| Fetal Bovine Serum (FBS) | Invitrogen | 26140079 | Cell culture medium additive |
| Pennicillin/ | Gibco | 15140-122 | Cell culture medium additive |
| streptomycin | | | |
| Chelex 100 | Fisher | C7901 | Removal of metals from water |
| Disposable column | Fisher | 731-1550 | Removal of chelex from water |
| Disposable 0.2 μm filter | Fisher | 09-740-39A | Removal of chelex from water |
| pSCN-Bn-DOTA | Macrocyclics | B-205 | Bifunctional chelator for ²²⁵Ac |
| Size exclusion spin column | Fisher | 89891 | Buffer exchange and |
| SG-TLC paper | Agilent | SGI0001 | Thin layer chromatography |
| Diethylenetriaminepentaacetic | Sigma | D1133-250G | Competitor/Solvent |
| acid | | | |
| Tetraethyl-ammonium- | Acros | AC420449250 | Buffer |
| acetate | | | |
| L-sodium ascorbate | Acros | AC3526800050 | Free radical scavenger |
| Phosphate-buffered saline | Fisher | BP399-4 | Buffer |
| Sodium chloride | Sigma | 46398-500G | Salt |
| Sodium acetate | Sigma | S7670-500G | Buffer |
| Ammonium acetate | Sigma | 41311-50G | Buffer |
| Ammonium | Sigma | 3733331 | Mass spectrometry |
| acetate 99.999% | | | |
| trace metal basis | | | |
| Human serum | Sigma | H4522 | Serum stability assay |
| 1.2 ml cluster tubes | Corning | 4413 | Cell viability assays |
| MTS (tetrazolium salt) | Promega | PR-3582 | MTS assay |
| colorimetric assay | | | |
| D-Luciferin | GoldBio | Luck-1G | Luciferase assay |
| Dark sided 96-well plates | Fisher | 07-200-627 | Luciferase assay |
| 1.5 ml tubes | Fisher | 07-200-244 | |
| 15 ml tubes | Fisher | 3144-355-001 | |

### DOTA Conjugation, ²²⁵Ac Chelation, Radiochemical Yield, Purity, and Mass Spectrometry

DOTA was dissolved in H₂O and conjugated to antibodies as previously described. For ²²⁵Ac chelation, 1.85 MBq of ²²⁵Ac in 0.2 M HCl was added to 2 M tetraethyl-ammonium-acetate to obtain pH 6.0. One hundred micrograms of DOTA antibody prepared at an 8:1 DOTA-to-antibody molar ratio were added, and the reaction was brought to 150 μL with 20 mM sodium acetate, 150 mM NaCl (pH 7.0), and 15 μL of a 10 mg/mL solution of sodium ascorbate. After 4 h at 37° C., diethylenetriaminepentaacetic acid (pH 7.0) was added to 5 mM final concentration for 10 min followed by size-exclusion column purification into saline with sodium ascorbate added to 10 μg/mL. All quantifications were at secular equilibrium, using a Capintec CRC 55tW dose calibrator and a Beckman 8000 γ-counter with a 250- to 480-keV energy window or by scanning of thin-layer chromatography strips. Fast protein liquid chromatography, thin-layer chromatography, and mass spectrometry were performed as previously described.

### Immunoreactivity, In Vitro Stability, and Cell Killing

Immunoreactivity was assayed by Raji-cell binding as previously described, without or with cold ofatumumab blocking. To assay stability, [²²⁵Ac]Ac-ofatumumab or ²²⁵Ac was added to human serum, incubated at 37° C., and assayed by thin-layer chromatography as previously described.

To assay cell killing, 5×10⁵ Raji-luciferase cells in 1 mL of RPMI medium with 10% heat-inactivated fetal bovine serum were added per well, followed by no antibody, native ofatumumab, [²²⁵Ac]Ac-IgG, or [²²⁵Ac]Ac-ofatumumab. When used, the antibody mass was 0.1 μg/well. Medium was exchanged after 24 h, and viability assays were performed 48 h later by MTS (3-(4,5-dimethylthiazol-2-yl)-5-(3-carboxymethoxyphenyl)-2-(4-sulfophenyl)-2H-tetrazolium) or bioluminescence imaging as previously described.

### Biodistribution in Tumor-Bearing Mice

The Washington University in St. Louis Institutional Animal Care and Use Committee approved the animal studies. Eight- to 10-wk-old R2G2 mice (no. 021; Envigo) were inoculated subcutaneously with 5×10⁶ Raji-Luc cells. When palpable tumors were present, 8-11 μg (4.07 kBq) of [²²⁵Ac]Ac-ofatumumab were administered intravenously and biodistribution analyzed 7 d later. The femur was measured after marrow extraction.

### Therapy and Bioluminescent Imaging

R2G2 mice were injected intravenously with 1×10⁶ Raji-Luc cells. In a first study, 8 d later, the mice either were untreated or were treated with ofatumumab or 3.7 or 9.25 kBq/mouse of [²²⁵Ac]Ac-IgG or [²²⁵Ac]Ac-ofatumumab. In a second study, 12 or 16 d after cell injection, the mice either were untreated or were treated with 9.25 kBq of [²²⁵Ac]Ac-ofatumumab. The administered antibody mass was adjusted to 20 μg/mouse of IgG or ofatumumab. Bioluminescent images were acquired and quantified as previously described and normalized to the first imaging time point. The mice were humanely euthanized if they had hind-limb paralysis (HLP), more than a 20% weight loss, or morbidity or reached the scheduled study termination point.

### Biodistribution and Dosimetry in Wild-Type Mice

Biodistribution and dosimetry studies were performed on 6- to 8-wk-old female C57BL/6N mice (no. 556; Charles River) injected intravenously with 3 μg (˜3.7 kBq) of [²²⁵Ac]Ac-ofatumumab. At selected time points after injection of [²²⁵Ac]Ac-ofatumumab, the mice were euthanized and organs γ-counted at secular equilibrium to determine decay-corrected percentage injected activity per gram (% IA/g). Bone (tibia and fibula) was counted after marrow separation.

Integrated time-activity curves from the murine data and the mean absorbed dose (D) were calculated according to MIRD methodology using the formula D=A×Δ×ϕ, where A is the cumulated activity, Δ is the mean α-particle energy, and ϕ is the absorbed fraction, with extrapolation to infinity, yielding a maximally conservative estimate. α particles were assumed to deposit all their energy locally (ϕ=1). The trapezoidal rule was used to integrate the time-activity curve of the α-particles emitted from the decay of ²²⁵Ac and its α-particle—emitting daughters (²²¹Fr, ²¹⁷As, ²¹³Bi, and ²¹³Po) using values from International Commission on Radiological Protection publication 107, with all daughter decays assumed to occur in the same organ as the ²²⁵Ac decay, yielding a Δ of 4.42⁻¹² J/(Bq·s) for ²²⁵Ac and its daughters. These mouse data were extrapolated to the adult female human model using the relative organ mass scaling method. Equilibrium in the decay chain and no translocation during the decay between succeeding disintegrations were assumed. Thus, the same estimated integrated time-activity curve obtained for ²²⁵Ac was applied to its daughters. The absorbed dose of [²²⁵Ac]Ac-ofatumumab was summed after applying weighting factors in the 2 possible pathways, 2% for ²⁰⁹Ti and 98% for ²¹³Po. A relative biological effectiveness of 5 for α-particles was used in the calculation of sieverts.

### Statistical Methods

GraphPad Prism software, version 8.4.3, was used for statistical analyses. A P value of less than 0.05 was considered statistically significant, with statistical tests noted in the text or figure legends. Data are shown as mean±SD.

### Results

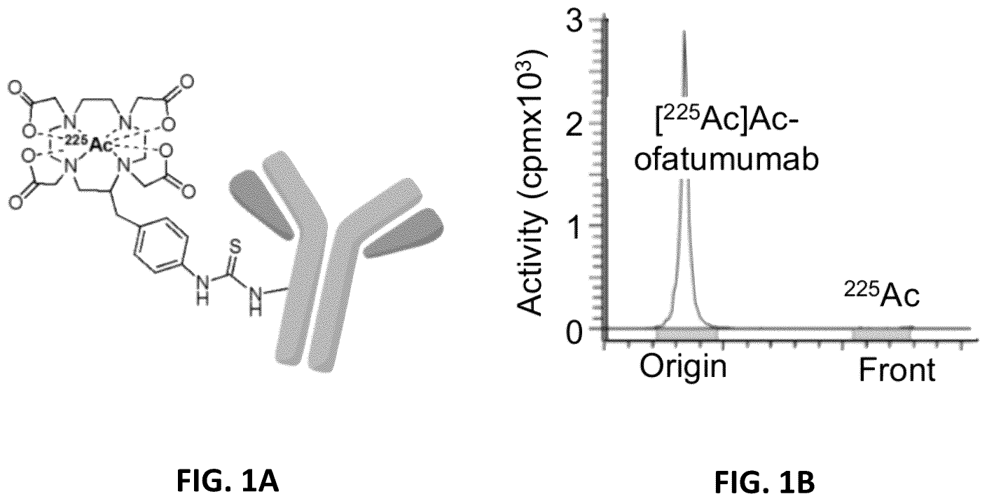

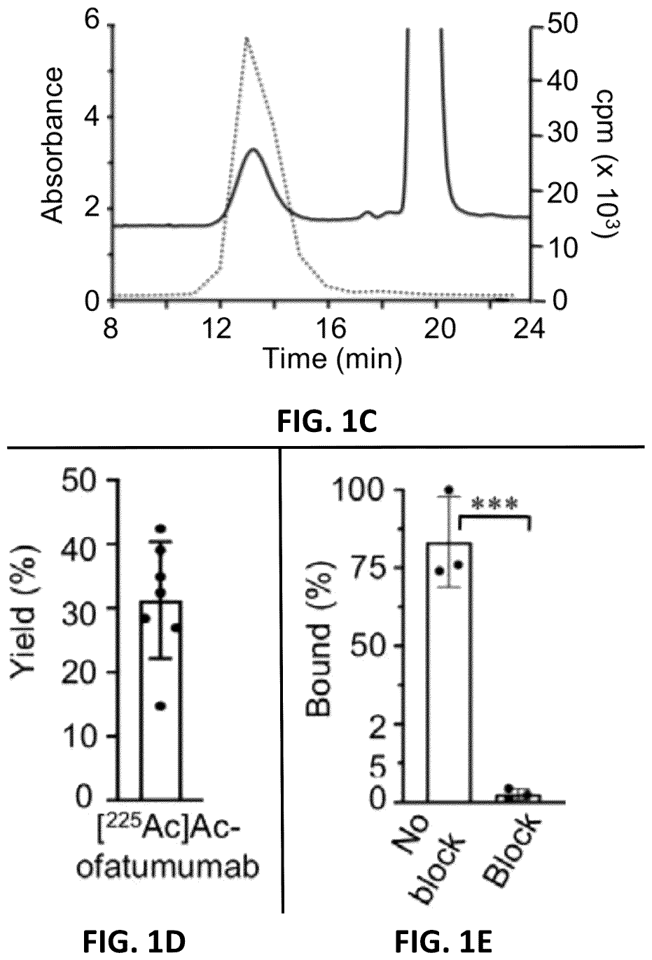

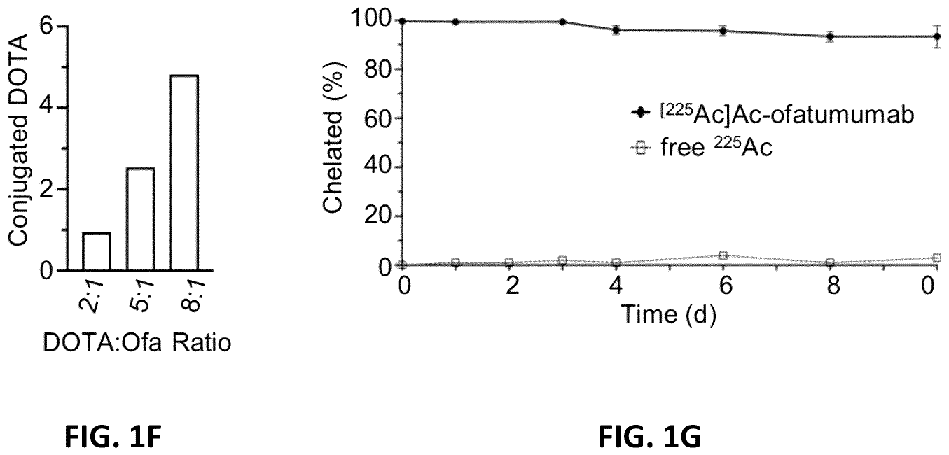

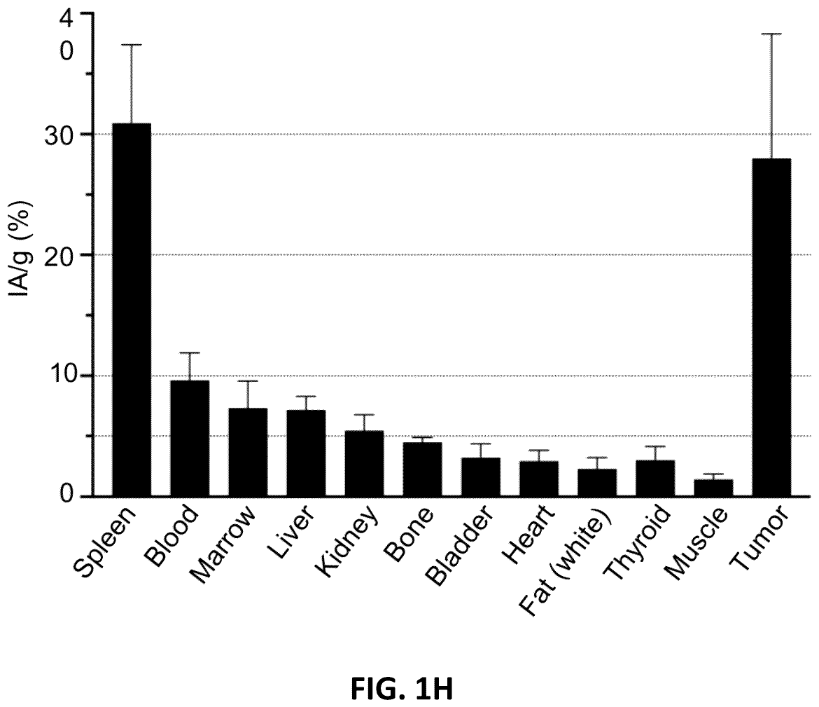

### [²²⁵Ac]Ac-Ofatumumab Synthesis, Radiochemical Yield and Purity, Chelate Number, Immunoreactivity, and Serum Stability

[²²⁵Ac]Ac-DOTA-ofatumumab was prepared using a 1-step method and characterized (FIG. 1(A-G)). Radiochemical purity was more than 95%, and radiochemical yields were more than 30%, with more than 70% immunoreactivity and specific activities of 5.25-15.6 MBq/mg. An average of 1, 2, and 5 chelates were attached per antibody using DOTA-to-ofatumumab ratios during conjugation of 2:1, 5:1, and 8:1, respectively. Chelation was stable after 10 d in serum.

Biodistribution with Subcutaneous Raji-Cell Tumors

Immunodeficient R2G2 mice were used (B6;129-Rag2^{tm1Fwa} II2rg^{tm1Rsky}/DwlHsd) that are proficient in double-strand DNA-break repair. Prkdc^{scid} mice that lack double-strand DNA repair because of the scid mutation are known to show artifactual radiosensitivity to DNA damage, such as that induced by α-particles. Compared with Prkde^{scid} mice, it was expected that the nontargeted (nontumor) cells in R2G2 mice will better reflect the response of nontargeted (nontumor) cells in humans to α-particle transit, as both tumor and nontumor cells are proficient in double-strand DNA-break repair.

The biodistribution of [²²⁵Ac]Ac-ofatumumab was evaluated 7 d after injection in mice bearing subcutaneous Raji tumors (FIG. 1H). The radioimmunotherapeutic showed a long circulatory residence time, consistent with stable chelation. This was confirmed by low liver uptake (7±1% IA/g), as free ²²⁵Ac shows high liver uptake. Splenic uptake was 31±6% IA/g, similar to that of [⁸⁹Zr]Zr-rituximab and -ofatumumab. Marrow and femur showed 7±3 and 5±0.05% IA/g, respectively. Tumor targeting was 28±10% IA/g.

### [²²⁵Ac]Ac-Ofatumumab-Mediated Cell Killing

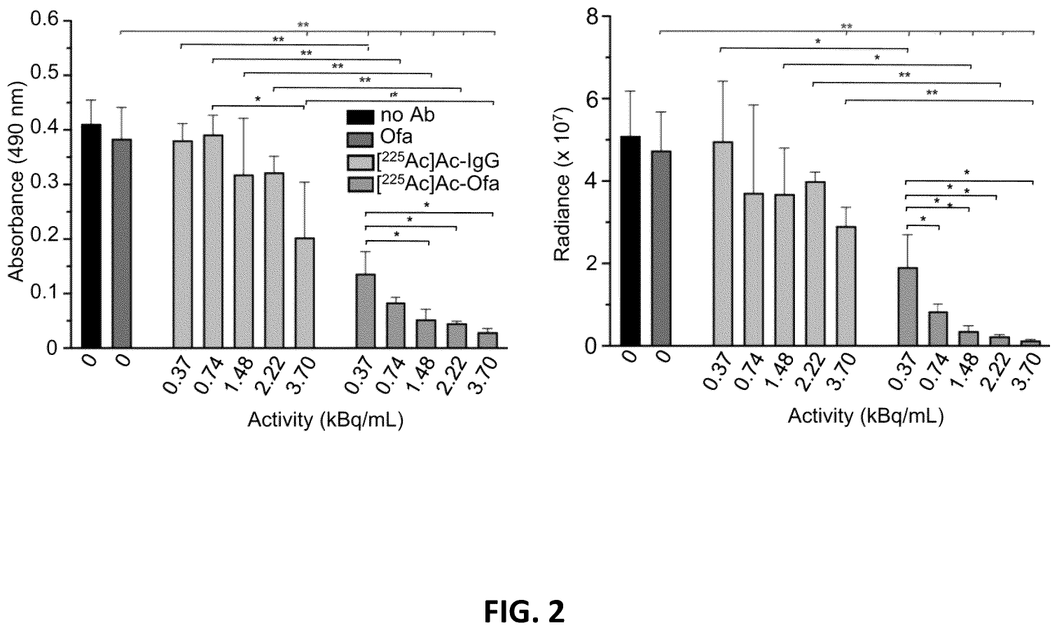

To investigate in vitro cytotoxicity, Raji-Luc cells were incubated with medium only, native (cold) ofatumumab, [²²⁵Ac]Ac-IgG, or [²²⁵Ac]Ac-ofatumumab for 24 h, followed by medium exchange and, 48 h later, viability assays (FIG. 2). Compared with no antibody, native ofatumumab did not affect viability. A 3.7 kBq/mL dose of [²²⁵Ac]Ac-IgG showed a modest (˜2-fold) effect, with none at lower doses. [²²⁵Ac]Ac-ofatumumab yielded significant, dose-dependent reductions in viability compared with cells without antibody or cells exposed to native ofatumumab or [²²⁵Ac]Ac-IgG.

### Biodistribution and Absorbed Dose of [225 Ac]Ac-Ofatumumab in C57BL/6N Mice

The biodistribution of [²²⁵Ac]Ac-ofatumumab was evaluated using non-tumor-bearing, wild-type C57BL/6N mice (TABLE 3). Blood [²²⁵Ac]Ac-ofatumumab levels were high (43±6% IA/g) at 4 h after injection and slowly fell to 15% IA/g at 12 d after injection. Uptake of ²²⁵Ac-ofatumumab was 5-8 IA/g in bladder, 8-11% IA/g in kidney, and 6-12% IA/g in marrow, with a gradual diminution in marrow over time. Liver uptake was 10-15% IA/g, with perhaps some contribution by dechelated ²²⁵Ac, which accumulates in the liver. Fecal ²²⁵Ac levels were consistent with [²²⁵Ac]Ac-ofatumumab or metabolites being excreted via the gastrointestinal route.

TABLE 3
Biodistribution of [225Ac]Ac-ofatumumab in C57BI/6N Mice. Values represent
mean % IA/g ± SD, except for urine and feces, which were collected
as a pooled sample. n = 5 mice per time point. Intestine, Lg L,
lower large intestine. Intestine, Lg U, upper large intestine nd, not done.

| Organ | 4 h | day 1 | day 2 | day 6 | day 12 |
|---|---|---|---|---|---|
| Adrenals | 6.68 ± 1.17 | 6.47 ± 1.49 | 5.12 ± 1.83 | 2.92 ± 2.06 | 1.53 ± 0.92 |
| Bladder | 5.75 ± 4.83 | 7.96 ± 1.46 | 6.74 ± 1.74 | 4.20 ± 1.18 | 5.02 ± 2.76 |
| Blood | 42.75 ± 5.98 | 24.20 ± 3.08 | 21.75 ± 4.52 | 16.18 ± 3.49 | 14.60 ± 2.71 |
| Bone | 4.92 ± 0.58 | 6.06 ± 0.67 | 6.83 ± 2.48 | 6.34 ± 1.71 | 6.98 ± 4.20 |
| Brain | 1.41 ± 0.28 | 0.73 ± 0.14 | 0.68 ± 0.13 | 0.55 ± 0.14 | 0.46 ± 0.03 |
| Fat | 2.54 ± 0.75 | 2.87 ± 0.52 | 1.86 ± 0.49 | 0.98 ± 0.65 | 1.42 ± 0.41 |
| Gallbladder | 5.75 ± 4.88 | 5.96 ± 2.62 | 5.19 ± 5.32 | 1.17 ± 0.84 | 2.92 ± 6.22 |
| Heart | 9.48 ± 1.35 | 5.85 ± 1.69 | 5.96 ± 1.42 | 4.68 ± 1.67 | 4.88 ± 1.21 |
| Intestine, | 4.00 ± 0.23 | 3.57 ± 0.72 | 2.99 ± 0.87 | 2.14 ± 0.46 | 1.55 ± 0.21 |
| Small | | | | | |
| Intestine, | 4.18 ± 0.54 | 3.67 ± 1.01 | 3.20 ± 0.85 | 2.51 ± 0.72 | 1.56 ± 0.08 |
| Lg, U | | | | | |
| Intestine, | 3.27 ± 0.51 | 2.42 ± 1.03 | 1.94 ± 0.24 | 1.31 ± 0.27 | 1.07 ± 0.25 |
| Lg, L | | | | | |
| Kidney | 10.62 ± 1.01 | 11.69 ± 4.93 | 8.29 ± 1.67 | 7.72 ± 1.03 | 6.85 ± 1.40 |
| Liver | 15.39 ± 2.21 | 14.45 ± 2.30 | 11.45 ± 1.11 | 13.81 ± 1.33 | 10.45 ± 1.44 |
| Lung | 13.91 ± 2.29 | 8.56 ± 1.42 | 7.39 ± 1.49 | 6.03 ± 1.36 | 6.50 ± 1.54 |
| Marrow | 12.98 ± 1.84 | 12.42 ± 2.78 | 15.17 ± 6.63 | 8.69 ± 2.59 | 6.58 ± 3.05 |
| Muscle | 2.07 ± 0.75 | 3.11 ± 0.33 | 2.76 ± 0.77 | 1.75 ± 0.61 | 1.82 ± 0.51 |
| Ovaries | 6.91 ± 2.03 | 5.18 ± 3.40 | 4.38 ± 0.81 | 3.15 ± 2.01 | 6.59 ± 5.76 |
| Pancreas | 4.26 ± 0.58 | 3.89 ± 0.64 | 4.08 ± 0.51 | 2.77 ± 1.00 | 3.05 ± 0.85 |
| Spleen | 15.39 ± 0.98 | 12.73 ± 0.87 | 12.96 ± 1.82 | 11.17 ± 2.10 | 10.80 ± 1.15 |
| Stomach | 2.06 ± 0.19 | 2.26 ± 0.45 | 1.33 ± 0.38 | 1.44 ± 0.40 | 0.96 ± 0.19 |
| Thymus | 5.24 ± 1.14 | 4.27 ± 0.85 | 4.77 ± 1.61 | 4.69 ± 1.36 | 3.45 ± 1.65 |
| Thyroid | 11.09 ± 2.91 | 5.65 ± 3.86 | 4.79 ± 0.91 | 3.55 ± 1.09 | 2.68 ± 1.15 |
| Urine | 10.28 | 6.25 | 11.75 | nd | nd |
| Feces | 1.68 | 15.14 | 23.08 | nd | nd |

[²²⁵Ac]Ac-ofatumumab human radiation dosimetry estimates were then determined for a human female model (TABLE 1). As free 213Bi in the kidney was not evaluated, this absorbed dose may be somewhat underestimated. Extrapolated human radiation dose estimates reveal heart wall (1,919 mSv/MBq) as the organ with the highest predicted dose, followed by liver, spleen, and red marrow at 1,833, 1,803, and 1,620 mSv/MBq, respectively. Marrow is likely the dose-limiting organ. The calculated effective dose equivalent was 1,496 mSv/MBq.

TABLE 1
Extrapolated Human Radiation Dose Estimates for
[²²⁵Ac]Ac-Ofatumumab. Mean absorbed doses
after injection of [²²⁵Ac]Ac-ofatumumab
into mice, extrapolated to adult human female model.

| Organ | Equivalent dose (mSv/MBq) |
|---|---|
| Adrenals | 400 |
| Bladder wall | 275 |
| Brain | 84 |
| Breast | 260 |
| Gallbladder | 235 |
| Heart wall | 1,919 |
| Intestine, small | 247 |
| Intestine, large, lower | 260 |
| Intestine, large, upper | 251 |
| Kidney | 1,187 |
| Liver | 1,833 |
| Lung | 1,076 |
| Marrow, red | 1,620 |
| Muscle | 313 |
| Ovaries | 943 |
| Pancreas | 507 |
| Skin | 260 |
| Stomach wall | 240 |
| Spleen | 1,803 |
| Thymus | 638 |
| Thyroid | 517 |
| Uterus | 1,446 |
| Effective dose | 883 |
| Effective dose equivalent | 1,496 |

### Therapeutic Evaluation in a Disseminated Model on Day 8 After Cell Injection

The magnitude, duration, and tumor targeting of the radiopharmaceutical in the biodistribution studies, as well as the in vitro tumoricidal activity, motivated an in vivo lymphoma treatment study. Therapeutic efficacy was evaluated in R2G2 mice with intravenously injected Raji-Luc cells, which become widely disseminated. This model recapitulates many features of clinical NHL, as it invades multiple organs, including the hematopoietic compartment. HLP is a frequent cause for censoring.

First, the maximal tolerated dose of [²²⁵Ac]Ac-ofatumumab in naïve R2G2 mice was identified. Eighty days after injection, 3.7, 11.1, 18.5, and 37 kBq/mouse yielded 5 of 5, 4 of 5, 3 of 5, and 0 of 5 survivors, respectively. Thus, for therapy single injections were used for the nonmyeloablative doses of 3.7 kBq/mouse (low dose) and 9.25 kBq/mouse (high dose).

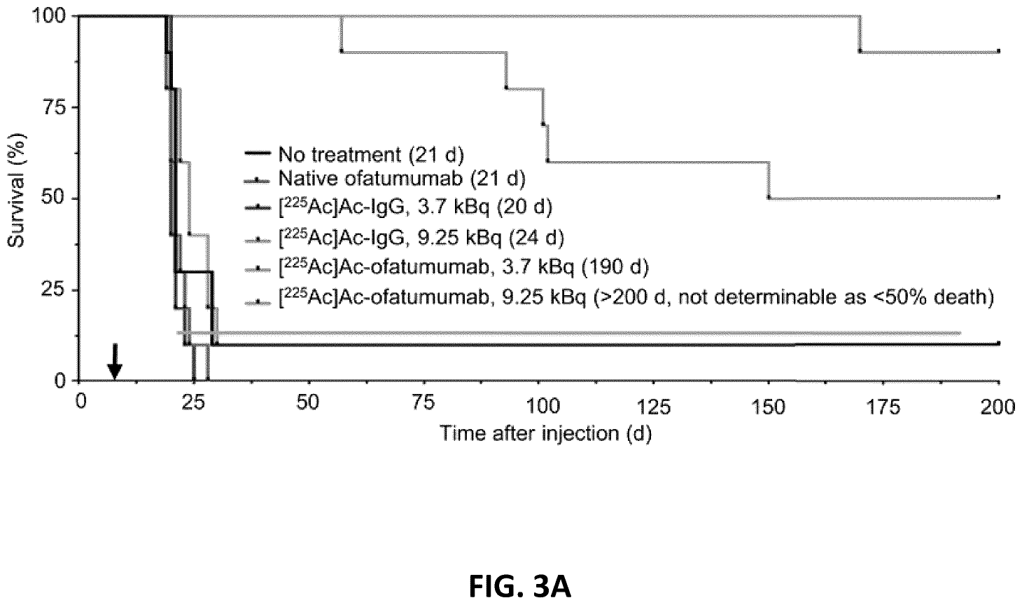

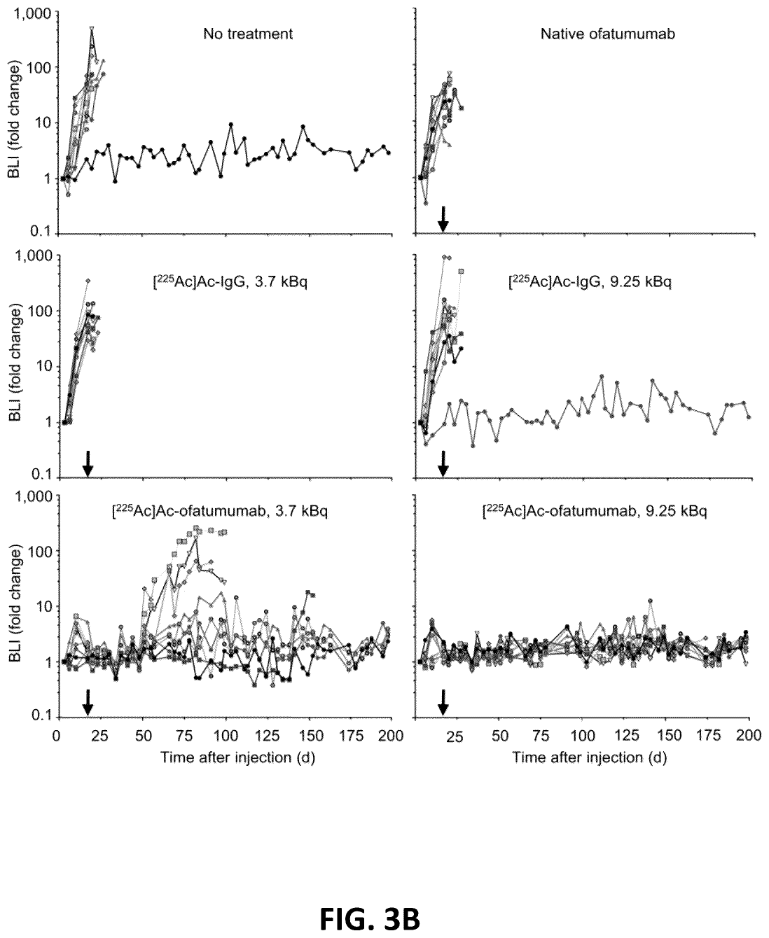

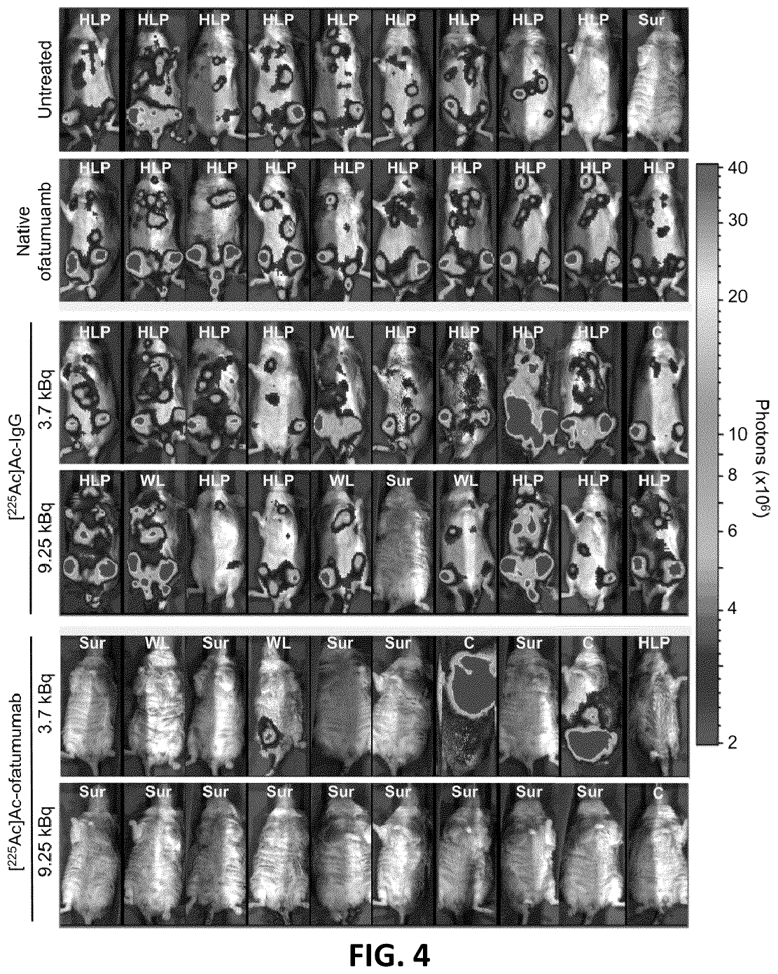

In the first therapy study, 8 d after cell injection the mice were randomized to remain untreated or to be treated with native ofatumumab or low or high doses of [²²⁵Ac]Ac-IgG or [²²⁵Ac]Ac-ofatumumab (n=10 mice per cohort). Survival, tumor burden, and weight were monitored for 200 d. In untreated mice, median survival was 21 d, with 9 of 10 mice succumbing before 29 d (FIG. 3A) and all mice showing increasing cancer-cell burden until censoring for HLP (FIG. 3B and FIG. 4). The lone surviving mouse never displayed cancer cells, suggesting an unsuccessful injection or engraftment. Native ofatumumab did not extend survival compared with untreated mice, with 10 of 10 succumbing before 29 d (FIG. 3A) and all showing increasing cancer-cell burden until censoring for HLP (FIG. 3B and FIG. 4).

Control radiolabeled nonspecific antibody, [²²⁵Ac]Ac-IgG, at low and high doses yielded median survivals of 20 and 24 d, respectively (FIG. 3A), which did not differ significantly from untreated or native-ofatumumab-treated mice (Mantel-Cox, P>0.05). All mice in the low-dose [²²⁵Ac]Ac-IgG cohort succumbed before 31 d. Nine were censored for HLP, and 1 perished (FIG. 4). In the high-dose cohort, 6 mice were censored for HLP and 3 for weight loss, with 1 surviving mouse that likely had an unsuccessful injection or engraftment (FIG. 3(A-B) and FIG. 4). All nonsurviving mice in both cohorts showed continuous cancer-cell growth until they were euthanized (FIG. 3B and FIG. 4).

In contrast, low- or high-dose [²²⁵Ac]Ac-ofatumumab treatment increased median survival to 190 d and more than 200 d (median survival was nondeterminable as there were <50% deaths), respectively (FIG. 3A), superior to survival of untreated or [²²⁵Ac]Ac-IgG-treated mice (Mantel-Cox, P<0.05). In the low-dose cohort, 5 of 10 mice survived. One mouse was censored for HLP, 2 for weight loss, and 1 for development of a leg tumor; 1 perished (FIG. 4). In the high-dose cohort, 9 of 10 mice survived, with 1 succumbing under anesthesia with no prior morbidity, weight loss, or detectable cancer cells (FIG. 3A and FIG. 4), indicating a death unrelated to disease or treatment. The median survival with high-dose [²²⁵Ac]Ac-ofatumumab was longer than that with low-dose [²²⁵Ac]Ac-ofatumumab (Mantel-Cox, P<0.05), indicating a dose-response effect.

All mice treated with low- or high-dose [²²⁵Ac]Ac-ofatumumab that survived had effective cancer-cell suppression with no detectable Raji-Luc cells at study termination (FIG. 3B and FIG. 4). The 5 nonsurviving mice in the low-dose cohort showed effective cancer-cell suppression for many days (˜50 d for 4 mice and ˜140 d for 1 mouse), followed by resumption of Raji-Luc cell proliferation, as indicated by increased bioluminescence (FIG. 3B and FIG. 4).

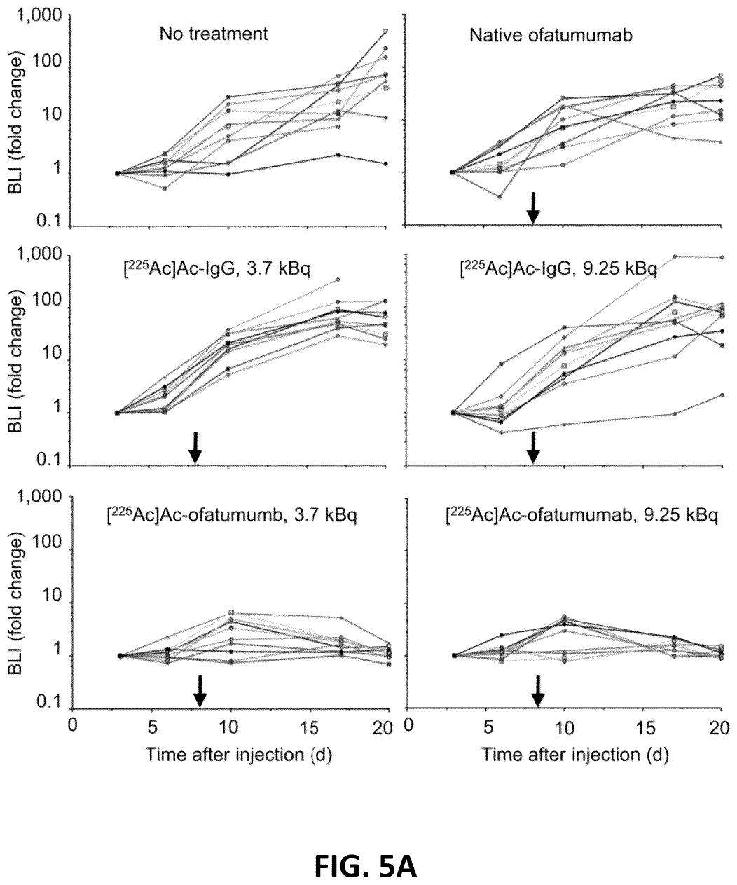

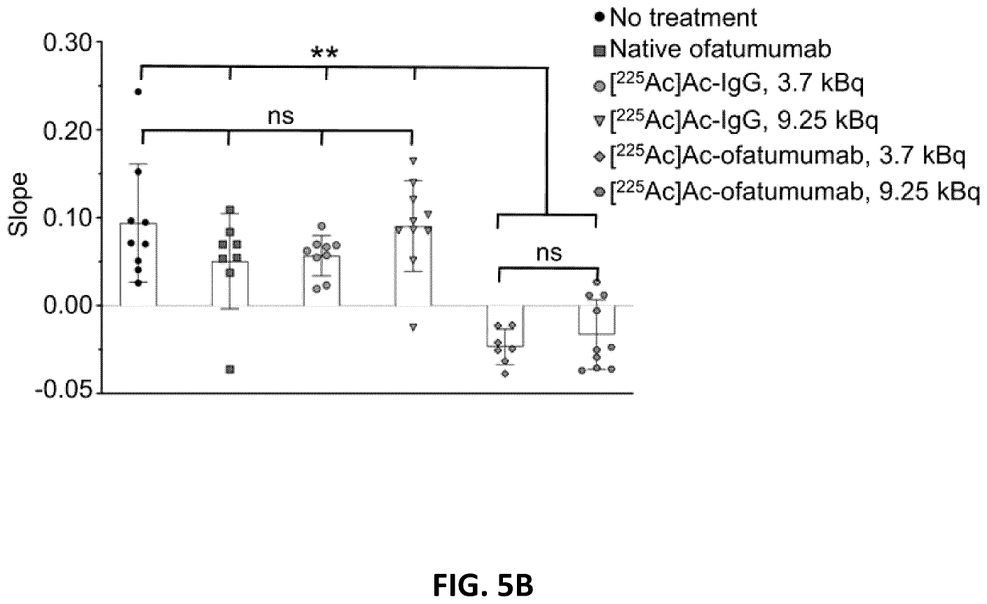

Because of its 10-d half-life, it was desirable to determine how rapidly [²²⁵Ac]Ac-ofatumumab treatment kills cancer cells. Comparison of bioluminescence from study day 3 to day 20 (5 d before and 12 d after starting therapy) revealed a continued increase in cancer-cell numbers in all cohorts except if treated with [²²⁵Ac]Ac-ofatumumab (FIG. 5A). To statistically test whether [²²⁵Ac]Ac-ofatumumab rapidly kills cancer cells in vivo, the log of the bioluminescence values from 10 to 20 d after cell injection (2-12 d after starting therapy) was plotted. Comparison of the slopes of these lines (FIG. 5B) confirmed no effect of native ofatumumab or [²²⁵Ac]Ac-IgG on cancer-cell proliferation. In contrast, both low- and high-dose [²²⁵Ac]Ac-ofatumumab significantly reduced these line slopes, indicating rapid killing of cancer cells after initiation of targeted therapy.

### Evaluation of Systemic Toxicity

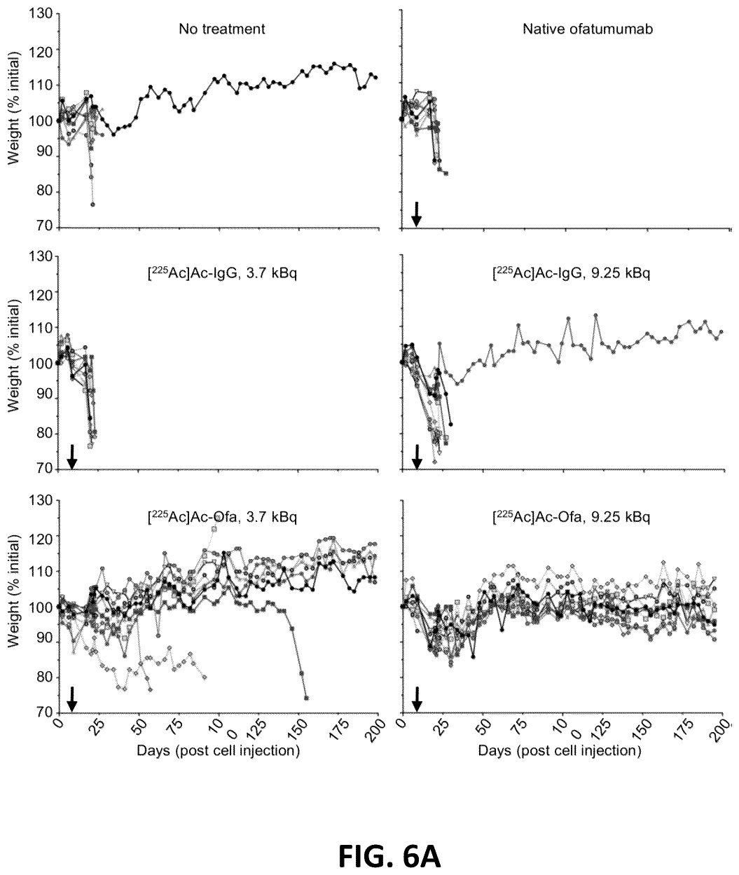

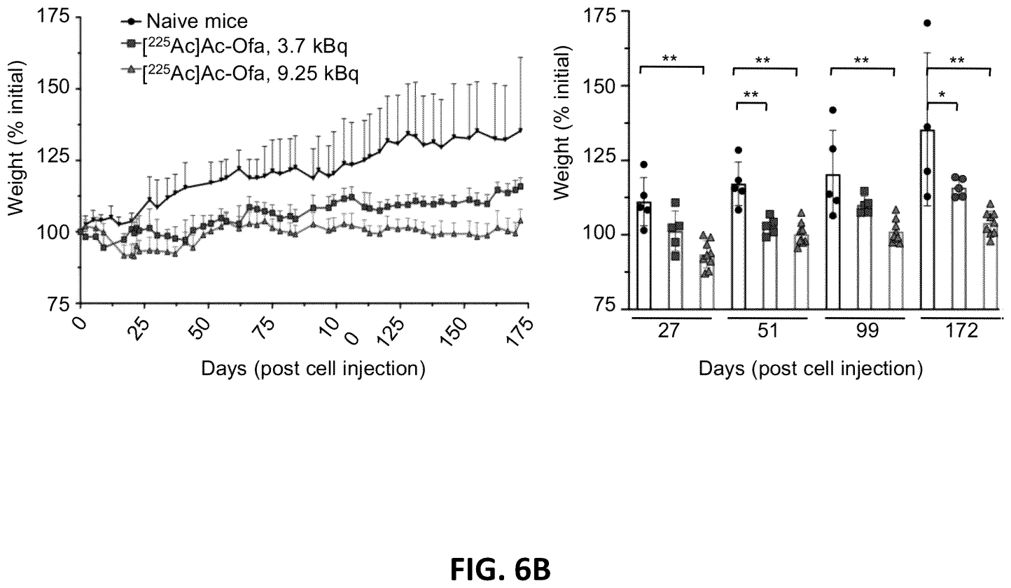

To investigate systemic toxicity of [²²⁵Ac]Ac-ofatumumab, animal weights in the therapy study initiated 8 d after cell injection were determined (FIG. 6(A-B)). Not surprisingly, most nonsurviving mice in all cohorts showed clear weight loss before they died or were euthanized for cause, consistent with the increased cancer-cell burden in these mice.

Mice in the low-dose [²²⁵Ac]Ac-ofatumumab-treated cohort that survived to study termination showed a continuous gradual weight gain (FIG. 6B, left). This was consistently slightly lower than the weight gain of control R2G2 mice that were not injected with cells or treated with therapy (FIG. 6B), with a significant difference present only after 172 d (FIG. 6B, right). Surviving mice in the high-dose [²²⁵Ac]Ac-ofatumumab cohort showed greater systemic effects, with initial loss of weight having a nadir at 27 d, followed by recovery to initial weight at 52 d and thereafter (FIG. 6B).

### Therapeutic Evaluation in a Disseminated Model on Days 12 and 16 After Cell Injection

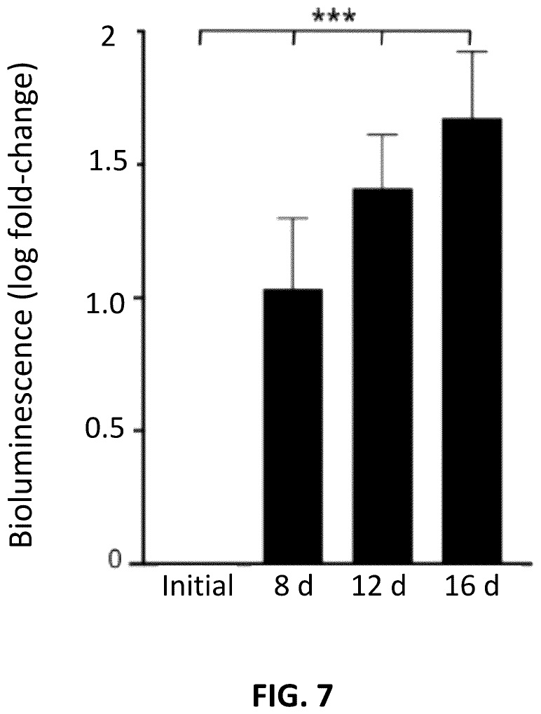

Next, it was determined whether tumor burden at the time of therapy is a relevant parameter in treatment outcome. Treatment was delayed from 8 d until either 12 or 16 d after cell injection (untreated mice typically die at 19-21 d after cell injection) to provide a larger pretreatment disseminated disease burden, comparing no treatment with high-dose (9.25 kBq) [²²⁵Ac]Ac-ofatumumab. To assay tumor-cell burden over time, bioluminescence in untreated mice 8, 12, and 16 d after cell injection was compared (FIG. 7) and confirmed a steadily increasing disease burden.

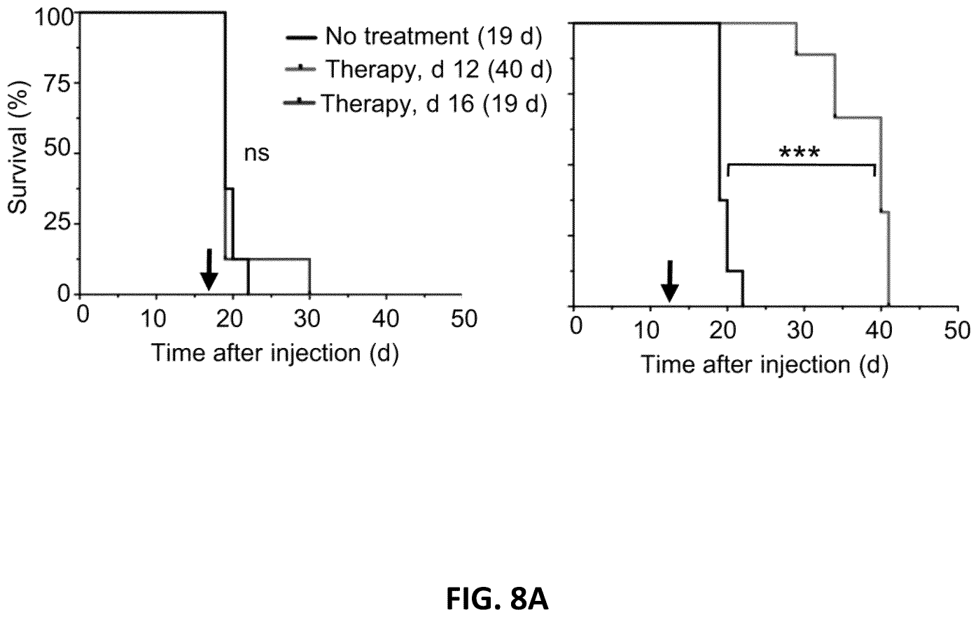

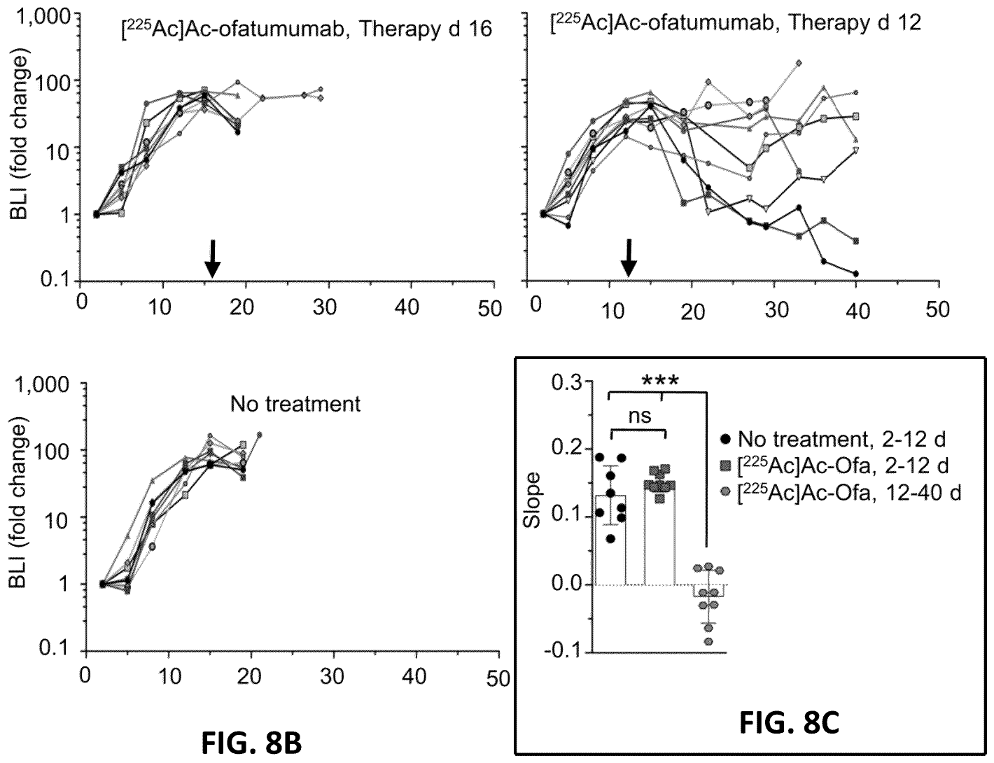

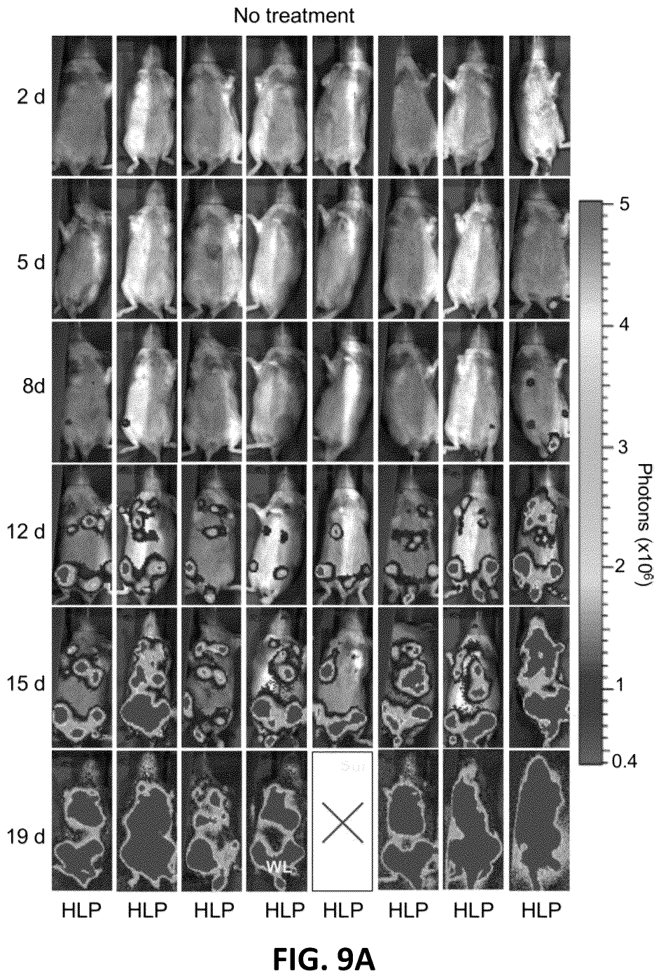

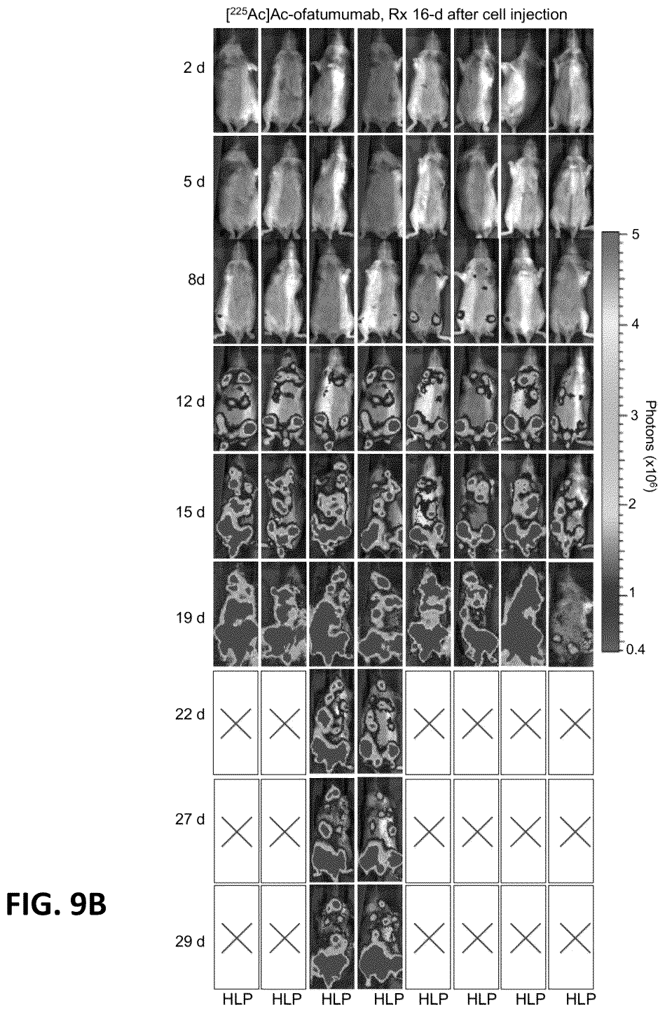

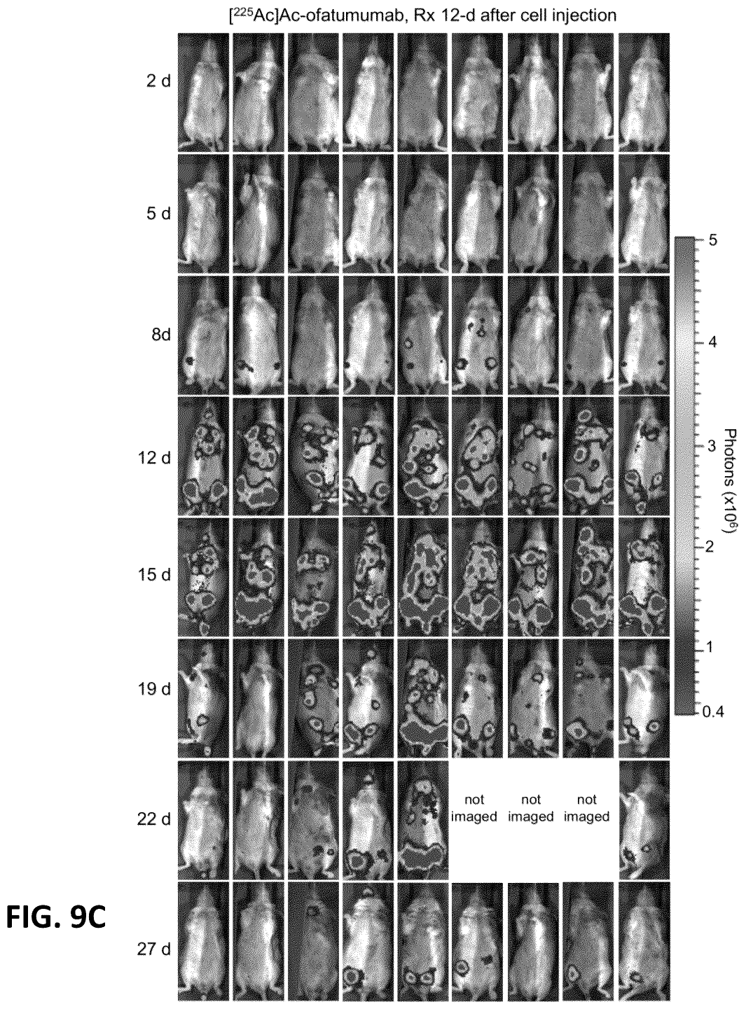

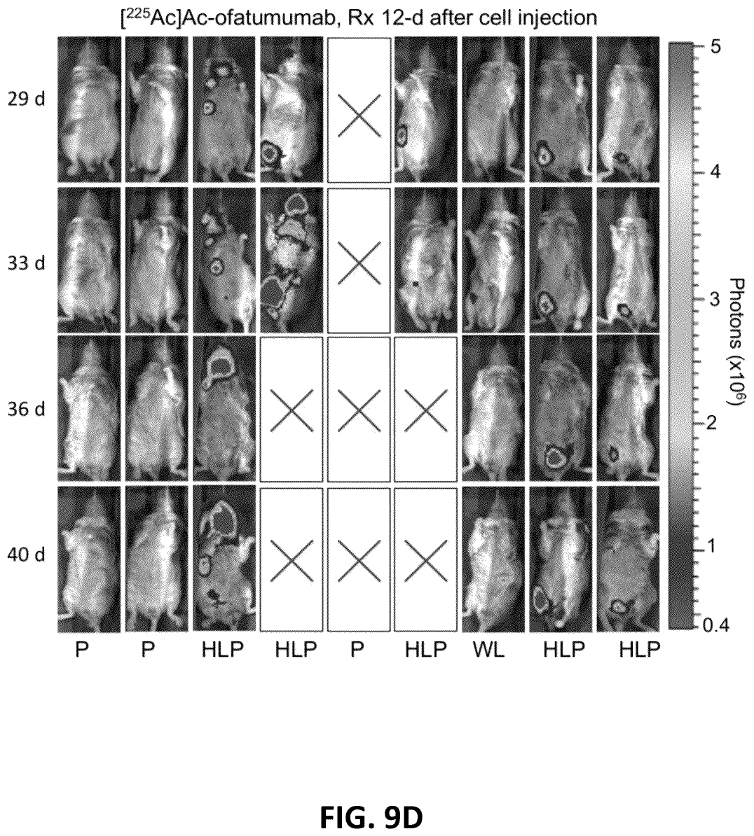

When therapy was initiated 16 d after cell injection, [²²⁵Ac]Ac-ofatumumab provided no survival benefit versus no treatment (FIG. 8A; median survival was 19 d for both cohorts (P>0.05, Mantel-Cox), with a high cancer-cell burden in both cohorts before censoring for HLP (FIG. 9A and FIG. 9B). Because the time of survival after treatment was very short, the effect on cancer-cell burden past the initiation of treatment was not sufficient for statistical analysis, but any effect appeared minimal (FIG. 8(A-C); FIG. 9(A-C)).

In contrast, a single treatment with [²²⁵Ac]Ac-ofatumumab 12 d after cell injection increased median survival to 40 d (FIG. 8A; Mantel-Cox, P<0.0001), although no mice survived beyond 41 d. This treatment prevented a further increase or yielded a reduction in cancer-cell burden (FIG. 8B and FIG. 8C; FIG. 9C and FIG. 9D). In this cohort, 5 mice were censored for HLP and 1 for weight loss; 3 perished (FIG. 9C and FIG. 9D).

### Discussion

The present disclosure further validates the use of anti-CD20 antibodies for radioimmunotherapy of NHL. [²²⁵Ac]Ac-ofatumumab was produced with high immunoreactivity, radiochemical yield, and purity and with stable chelation of ²²⁵Ac in vitro and in vivo. [²²⁵Ac]Ac-ofatumumab specifically killed CD20-expressing cells in vitro and showed high tumor uptake in vivo. In therapeutic studies using a murine model of disseminated human lymphoma, a single [²²⁵Ac]Ac-ofatumumab treatment showed excellent efficacy, with curative ability except for very advanced disease.

[²²⁵Ac]Ac-ofatumumab was prepared using a mild 1-step procedure in which ²²⁵Ac is directly chelated to DOTA-conjugated antibody at 37ºC. Other investigators also have used this approach. ²²⁵Ac was stably chelated, and uptake of [²²⁵Ac]Ac-ofatumumab uptake by CD20-expressing subcutaneous tumors (28% IA/g) was similar to the 33% IA/g of [⁸⁹Zr]Zr-DFO-ofatumumab. The approximately 5 MBq/mg specific activity obtained for [²²⁵A]Ac-ofatumumab was sufficient for preclinical use and should enable scaling up for clinical use.

The major finding of the current study was the high therapeutic potency of [²²⁵Ac]Ac-ofatumumab in mice with an aggressive murine model of disseminated human lymphoma, similar to micrometastatic disease in humans. When administered 8 d after cell injection, control unlabeled ofatumumab and low (3.7 kBq/mouse) or high (9.25 kBq/mouse) doses of nontargeted [²²⁵Ac]Ac-IgG did not improve median survival compared with untreated mice or inhibit cancer-cell growth. In contrast, both low and high doses of [²²⁵Ac]Ac-ofatumumab rapidly and significantly inhibited cancer-cell growth and increased median survivals to 190 and more than 200 d, respectively. Half the mice in the low-dose group survived, and none of the animals in the high-dose group had disease- or treatment-related mortality. All mice in these 2 cohorts that survived until study termination showed apparent complete elimination of cancer cells, as no cancer cells were detected at study completion on day 200. Also, the potential for systemic anticancer effects was tested in disease settings of further progression. Twelve days after cell injection, high-dose [²²⁵Ac]Ac-ofatumumab killed cancer cells and significantly extended median survival to 40 d, showing therapeutic benefit even with a high pretreatment burden of cancer cells, although the therapy was not curative. Not surprisingly, there was a limit to therapeutic efficacy; therapy initiated 16 d after cell injection was unable to improve survival. This lack of benefit was likely because approximately 3 d before expected death was not enough time to allow for sufficient targeting and absorbance of the dose of intact radiolabeled antibody, and possibly also because of altered pharmacokinetics due to the high cancer-cell burden.

Dosimetry of organs from [²²⁵Ac]Ac-ofatumumab-injected C57BL/6N mice extrapolated to an adult female human model suggests that marrow will be the dose-limiting organ, as is common with unsealed, intact antibody-based radiotherapeutics, including Bexxar and Zevalin. It is worth noting that uptake in nontumor target organs can often be significantly reduced by predosing with unlabeled antibody, which improves lesion-to-background-organ ratios, as used in the Zevalin and Bexxar therapeutic regimens and in other applications. Fully human ofatumumab is unlikely to induce an immune response, increasing the potential for fractionated dosing of [²²⁵Ac]Ac-ofatumumab to ameliorate myelotoxicity. In addition, autologous stem-cell transplantation after radioimmunotherapy, as used in some Zevalin and Bexxar protocols, may be useful. Development of a theranostic partner, such as [⁸⁹Zr]Zr-ofatumumab, for personalized image-based dosimetry is also considered herein.

Notably, low-dose [²²⁵Ac]Ac-ofatumumab treatment 8 d after cell injection was curative in half the mice, which displayed weight gains similar to those of control naïve mice without tumor cells or therapy. The 5 nonsurviving mice in this cohort showed effective repression of cancer-cell growth for approximately 50-140 d and gains of weight over this time of remission. Thus, fractionated cycles of this low-dose therapy may induce cancer-cell elimination, as well as curative efficacy in even more mice while maintaining reduced toxicity. Mice treated with high-dose [²²⁵Ac]Ac-ofatumumab showed reduced weight gains compared with naïve mice, indicating some systemic toxicity. Some of this may result from kidney distribution of the ²²⁵Ac daughter radionuclide, ²¹³Bi, although this possibility remains to be confirmed. However, if so, there are approaches that may ameliorate such toxicity.

As described herein, the therapeutic efficacy of β-particle-emitting [¹⁷⁷]Lu-ofatumumab was tested using the murine disseminated Raji-Luc lymphoma model of the present disclosure. When therapy was started 4 d after cell injection, 8.51 MBq of [¹⁷⁷Lu]Lu-ofatumumab showed remarkable therapeutic efficacy, with apparent complete elimination of tumor cells and no disease-related deaths. As disclosed herein, it was further indicated that when therapy was initiated 8 d after cell injection, [¹⁷⁷Lu]Lu-ofatumumab was ineffective, indicating potential superiority of [²²⁵Ac]Ac-ofatumumab under this condition. Similar observations were noted previously using a murine disseminated multiple-myeloma model, comparing [¹⁷⁷Lu]Lu- and [²²⁵Ac]Ac-daratumumab.

Therapeutic efficacy of rituximab (an internalizing anti-CD20 antibody, like ofatumumab) radiolabeled with ²¹³Bi (half-life [t½], 45.6 min; 1 net α-particle) has been reported, applying a similar disseminated Raji-Luc model using Prkdc^{scid} mice. Cancer-cell killing was effective, and cures were common when the single-dose treatment was started 4 d after injection of 1×10⁶ Raji-Luc cells. However, single-dose [²¹³Bi]Bi-rituximab treatment was much less effective when initiated 7 d after cell injection, being markedly less effective than single-dose treatment with [²²⁵Ac]Ac-ofatumumab initiated 8 d after cell injection as found in the current study, although in a somewhat different animal system. These results may in part relate to the decay rates of ²¹³Bi versus ²²⁵Ac and the localization times for intact antibodies to tumor. However, if targets are readily accessible, ²¹³Bi can deliver a high dose rate, but if there exist groups of cells, or even solid tumors, with reduced antibody accessibility, sufficient penetration or dose deposition may not occur before radioactive decay, rendering the therapy less effective and yielding superior results with the longer-lived ²²⁵Ac. In this view, for antibody-mediated radioimmunotherapy for larger tumors, ²²⁵Ac may be a more appropriate choice for α-particle-mediated therapy. Further evaluation of the comparative therapeutic and off-target effects of ²¹³Bi-versus ²²⁵Ac-labeling for radioimmunotherapy with intact antibodies is of interest.

Other investigators have studied α-emitters for radioimmunotherapy of NHL using preclinical models. Previous therapeutic benefit had been found using a pretargeted approach with a ²¹³Bi-labeled anti-CD20 1F5(ScFv)₄SA molecule. Similarly, [²¹¹At]At-1F5 (²¹¹At t½, 7.2 h; 1 net α-particle) was 80% curative 6 d after intravenous cell injection with supporting stem cell transplantation but only poorly effective on subcutaneous tumors, perhaps limited by radioactive decay before tumor penetration. [²¹²Pb]Pb-rituximab (²¹²Pb t½, 10.6 h; 1 net α-particle) in a disseminated model improved survival times with therapy initiated at both low- and high-tumor burdens. [²²⁷Th]Th-rituximab (²²⁷Th t½, 18.7 d; 5 net α-particles) was often curative of small subcutaneous tumors and was superior to [⁹⁰Y]Y-ibritumomab-tiuxetan, although targeting of the ²²³Ra daughter to bone adds complexity. Similarly, treatment with [¹⁴⁹Tb]Tb-rituximab (¹⁴⁹Tb t½, 4.2 h, 1 α-particle) shortly after intravenous injection of Daudi cells significantly increased survival. The surprising and excellent results with ²²⁵Ac labeling disclosed herein likely reflect a very good match between antibody-specific targeting times to tumor and the half-life of this α-particle emitter. In conclusion, a single dose of [²²⁵Ac]Ac-anti-CD20 ofatumumab showed excellent therapeutic efficacy in a murine model of human disseminated lymphoma and was often curative. Fractionated dosing may improve efficacy. With increasing use of targeted radiotherapies, a renewed application of radioimmunotherapy targeting CD20 for NHL seems warranted, given the exceptional therapeutic efficacy of [²²⁵Ac]Ac-anti-CD20 ofatumumab and remaining unmet clinical needs in this disease.

### CONCLUSION

[²²⁵Ac]Ac-ofatumumab shows good in vitro characteristics and effectively targets CD20-expressing tumor xenografts. [²²⁵Ac]Ac-ofatumumab showed curative efficacy in a murine model of disseminated human lymphoma.

### Example 2: Cure of Disseminated Human Lymphoma with [177Lu]Lu-Ofatumumab in a Preclinical Model

This Example describes a novel therapeutic approach for treating cancer and other diseases, including lymphoma and MS, with [177Lu]Lu-Ofatumumab.

Non-Hodgkin lymphoma (NHL) is a common hematologic malignancy, with over 80,000 new cases and 20,000 deaths estimated for the United States in 2022 (1). The standard of care for many cases of NHL involves chemotherapy and immunotherapy targeting the CD20 protein, which is highly expressed on most NHL cells, with murine/human chimeric rituximab used most commonly. Although this chemotherapy-with-immunotherapy combination is usually initially effective, many cases are refractory or undergo relapse, indicating the need for improved therapies.

Radioimmunotherapy joined clinical practice 2 decades ago with Food and Drug Administration approval of 2 anti-CD20 radioimmunotherapies for lymphoma: Zevalin ([⁹⁰Y]Y-ibritumomab tiuxetan; Acrotech Biopharma, Inc.) and Bexxar (tositumomab and ¹³¹I-tositumomab; GlaxoSmithKline), which use murine-derived anti-bodies radiolabeled with b-particle-emitting radioisotopes. Because of potential immune reactions, these antibodies were approved for only a single therapeutic dose. ⁹⁰Y (half-life [t_{1/2}], 2.7 d) emits high-energy β-particles (average, 934 keV), whereas ¹³¹I emits lower-energy β-particles (average, 187 keV), with average ranges in tissue of 3,800 mm and 360 mm, respectively, enabling killing over many cell diameters. Thus, in addition to working against individual tumor cells, b-particles may work against larger tumors, tumor-cell aggregates with imperfect antibody access, and heterogeneous tumors, although with potential off-target damage. Despite long-term safety and clinical effectiveness, Bexxar has been discontinued in the United States and Zevalin is applied infrequently, in part because of economic and logistic concerns that were present when they were introduced and because of competing nonradioactive therapies.

Some concerns that limited the use of Bexxar and Zevalin have been overcome with greater integration of radiopharmaceutical therapy into medicine (5), as exemplified by the Food and Drug Administration approval of ¹⁷⁷Lu-labeled agents for prostate cancer treatment (Pluvicto; Advanced Accelerator Applications (6)) and neuroendocrine tumors (Lutathera; Advanced Accelerator Applications (7)). ¹⁷⁷Lu (t_{1/2}, 6.6 d) emits β-particles of 149 keV on average, with an average tissue range of 220 μm. Emission of low-abundance γ-particles by ¹⁷⁷Lu permits imaging by SPECT.

Recently, ofatumumab, a third-generation anti-CD20 fully human antibody, was developed. Ofatumumab is a type I antibody that is internalized after CD20 binding (8). We showed by biodistribution and PET imaging studies that [⁸⁹Zr]Zr-DFO-ofatumumab targets CD20-positive subcutaneous xenograft tumors as well as [⁸⁹Zr]Zr-DFO-rituximab (9).

Here, we describe the synthesis and evaluation of [¹⁷⁷Lu]Lu-ofatumumab. We present in vitro characteristics, dosimetry estimation, and subcutaneous tumor targeting. We also show that [¹⁷⁷Lu]Lu-ofatumumab therapy results in long-term survival and elimination of tumor cells in a murine model of disseminated human lymphoma.

### Materials and Methods

### Reagents and Cell Culture

Ofatumumab (IgG1 κ; Novartis) was purchased from the Washington University clinical pharmacy, and human IgG1 κ was purchased from BioXcel. Raji cells and Raji-luc cells stably expressing luciferase (10) were cultured as previously described (9). SCN-CHX-A″-DTPA ({[(R)-2-amino-3-(4 isothiocyanatophenyl)propyl]-trans(S,S)-cyclohexane-1,2-diamine-pentaacetic acid}) was from Macrocyclics, size-exclusion chromatography columns from Fisher Scientific, and d-luciferin from GoldBio. Sigma provided human serum, sodium acetate, diethylenetriamine pentaacetate, tetramethylammonium acetate, and I-sodium ascorbate. 177Lu from the University of Missouri was dissolved in 0.2 M HCl. Silica gel thin-layer chromatography paper was from Agilent, and the 3-(4,5-dimethylthiazol-2-yl)-5-(3-carboxymethoxyphenyl)-2-(4-sulfophenyl)-2H-tetrazolium (MTS) salt assay was from Promega.

### Antibody Conjugation, Radiolabeling, Thin-Layer Chromatography, Mass Spectrometry, and Fast-Performance Liquid Chromatography

Antibody was incubated with SCN-CHX-A″-DTPA in 0.1 M sodium carbonate, pH 9.0, at a chelator-to-antibody molar ratio of 8:1 for 1 h at 37° C. and purified by size-exclusion chromatography into 0.5 M NH₄OAc, pH 7.0. A 477-MBq quantity of ¹⁷⁷Lu was added to 400 μg of CHX-A″-DTPA-antibody with 20 mM NH₄OAc, pH 7.0. After 2 h at 37° C., DTPA was added to 5 mM final concentration, followed by size-exclusion chromatography purification into saline and the addition of a 10 mg/mL concentration of I-sodium ascorbate. Thin-layer chromatography and fast-performance liquid chromatography were done as previously described (9). Radiochemical yield was assayed with a CRC55-tW dose calibrator. Chelate number was determined using a Fisher Scientific Exactive Plus EMR mass spectrometer operating at a mass (m)-to-charge (z) range from 800 to 12,000 and a resolving power of 8,750 or 17,500 at 300 m/z. Data were analyzed using Protein Metric Intact software.

### Serum Stability, Immunoreactivity, In Vitro Stability, Affinity, and Cell Killing Assays

To assay stability, 14.8 MBq of [¹⁷⁷Lu]Lu-ofatumumab or ¹⁷⁷Lu were added to 10% human serum in 20 mM NaOAc 150 mM NaCl pH 7.0 with 10 mg/mL L-SA and incubated at 37° C. Another aliquot of [¹⁷⁷Lu]-ofatumumab was incubated at 4° C. in buffer without serum and with 10 mg/mL L-SA. Aliquots were analyzed by thin-layer chromatography at 0, 1, 5, and 7 d. Immunoreactivity was assayed as previously described (9). To assay affinity, 2.5×10⁶ Raji cells without or with 10 μg of ofatumumab were incubated with [¹⁷⁷Lu]Lu-ofatumumab, washed after 4 h at 23° C., and γ-counted. To assay cell killing, 2×10⁶ Raji-luc cells in 1 mL of RPMI medium with 10% heat-treated fetal bovine serum were exposed to no treatment, ofatumumab, [¹⁷⁷Lu]Lu-IgG, or [¹⁷⁷Lu]Lu-ofatumumab, with cognate unlabeled antibody added to 20 μg total. After 14 h at 37° C., the cells were washed and 20% were resuspended in fresh medium for an additional 168 h followed by MTS assay.

Biodistribution of [¹⁷⁷Lu]Lu-Ofatumumab in Mice with Subcutaneous Raji Tumors

The Washington University in St. Louis Animal Care and Use Committee approved the animal studies. Biodistribution with tumor-bearing mice used female 6- to 8-wk-old immunodeficient Rag2-IL2rg (R2G2, B6; 129-Rag2^{tm1Fwa}II2rg^{tm1Rsky}/DwlHsd) mice (Envigo) injected subcutaneously with 5×10⁶ Raji-luc cells. Mice with palpable tumors were injected intravenously with 10-20 μg of [¹⁷⁷Lu]Lu-ofatumumab and killed 1, 3, or 7 d later. Distribution was calculated as decay-corrected percentage injected activity per gram (% IA/g) using a Beckman 8000 γ-counter and a 1- to 500-keV window.

### Dosimetry Estimation

Naïve 5- to 6-wk-old C57BI6/N mice injected intravenously with 370 kBq (10 μg) of [¹⁷⁷Lu]Lu-ofatumumab were killed 4 h, 1 d, 2 d, 5 d, or 11 d later, and tissue and organs were γ-counted. Bone was counted after marrow separation. Urine and feces were collected at 4 h, 1 d, and 2 d. Organ residence times were calculated by analytic integration of single or multiexponential fits of the time-activity curve and scaled to human organ weight by relative organ mass scaling (11), which was not applied to the gastrointestinal tract organs. To estimate human radiation dose, residence times were entered into OLINDA, version 2.2, using the MIRD adult-female model and organ weights from International Commission on Radiological Protection publication 106 (12). The calculated radiation dose includes contributions from β- and γ-rays from ¹⁷⁷Lu within the organ, neighboring organs, and remainder of the body.

### Therapeutic Studies, Mouse Weight, and Bioluminescent Imaging

R2G2 mice (10 per group) injected intravenously with 1×10⁶ Raji-luc cells and either left untreated or injected 4 d later with ofatumumab, [¹⁷⁷Lu]Lu-IgG, or [¹⁷⁷Lu]Lu-ofatumumab. When used, 20 μg of antibody were injected per mouse. Bioluminescent images were acquired as previously described (13). Mice were killed if they experienced hind-limb paralysis, lost more than 20% of their body weight, or had other signs of morbidity.

### Statistics

Statistical analyses used Prism software (version 9.0; GraphPad).

### Results

### Synthesis of [¹⁷⁷Lu]Lu-Ofatumumab and Radiochemical Yield, Purity, and Immunoreactivity

SCN-CHX-A″-DTPA was conjugated to ofatumumab and purified. Mass spectrometry indicated an average of 3.2 chelators per antibody. After ¹⁷⁷Lu radiolabeling, [¹⁷⁷Lu]Lu-ofatumumab was purified (n=7). Radiochemical purity was more than 99%±1%, radiochemical yield was 93%±2%, and specific activity was 401±17 MBq/mg. Immunoreactivity was 49%±3% and 2%±1% after blocking with unlabeled ofatumumab.

### Serum Stability, In Vitro Cell Killing, and Affinity of [¹⁷⁷Lu]Lu-Ofatumumab

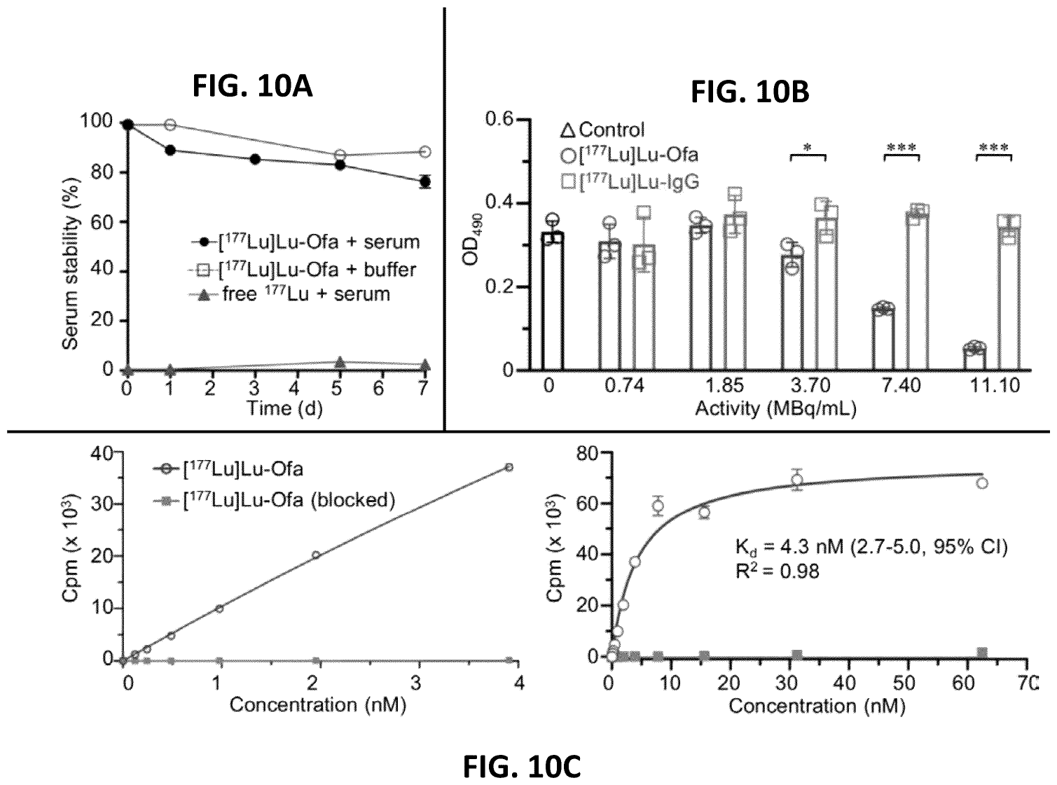

After 7 d, over 90% of 177Lu remained chelated in buffer at 4° C., and over 75% remained chelated in human serum at 37° C. (FIG. 10A; Targeting and killing of CD20-expressing cells were assayed (FIG. 10B) by adding either no antibody or [¹⁷⁷Lu]Lu-ofatumumab or [¹⁷⁷Lu]Lu-IgG (0.74-11.10 MBq/mL) to Raji-luc cells; incubating for 14 h; changing the medium; and, 168 h later, determining cell viability. Compared with no antibody, [¹⁷⁷Lu]Lu-IgG showed no cell killing at any dose. [¹⁷⁷Lu]Lu-ofatumumab at 3.7 MBq/mL or higher showed dose-dependent killing compared with no antibody and [¹⁷⁷Lu]Lu-IgG (P<0.05, n=3). [¹⁷⁷Lu]Lu-ofatumumab showed a 4.3 nM dissociation constant for CD20 (FIG. 10C), consistent with that noted previously (as described for 2F2 by Teeling et al. (14)).

### Biodistribution of [¹⁷⁷Lu]Lu-Ofatumumab in C57BI6/N Mice and Estimation of Human Dosimetry

[¹⁷⁷Lu]Lu-ofatumumab biodistribution was determined in C57BI6/N mice 4 h, 1 d, 2 d, 7 d, and 11 d after injection (TABLE 4) as % IA/g. Blood % IA/g was 38% at 4 h and 19% after 11 d. Bone distribution was less than 4%, indicating stable chelation because free 177Lu is a bone-seeking radionuclide (15). Liver was 9% IA at 4 h and 5% IA/g at 11 d, and marrow was 14% IA at 4 h and 9% IA/g at 11 d. Spleen was 8-9% IA/g. Approximately 13% of the injected activity was excreted.

TABLE 4
Biodistribution (% IA/g) of [177Lu]Lu-ofatumumab in C57BI/6N mice.
Values represent % IA/g ± SD, except for urine and feces, which
were collected as a pooled sample. n = 5 mice/time point. Sm Int,
small intestine. U Lg Int, upper large intestine. L Lg Int, lower large intestine.

| [₁₇₇Lu]Lu- | | | | | |
|---|---|---|---|---|---|
| ofatumumab | 4 h | 1 d | 2 d | 7 d | 11 d |
| Blood | 37.63 ± 7.76 | 26.57 ± 1.76 | 31.04 ± 2.24 | 23.37 ± 4.04 | 19.22 ± 2.77 |
| Heart | 10.18 ± 3.12 | 7.6 ± 1.81 | 8.22 ± 1.76 | 6.24 ± 1.14 | 5.37 ± 1.32 |
| Liver | 9.07 ± 1.88 | 6.36 ± 0.50 | 6.89 ± 0.98 | 5.96 ± 0.93 | 5.30 ± 0.72 |
| Bone | 3.32 ± 0.73 | 3.59 ± .29 | 3.31 ± 0.49 | 3.81 ± 0.37 | 3.36 ± 0.41 |
| Marrow | 13.88 ± 2.26 | 12.20 ± 1.55 | 14.84 ± 3.91 | 9.63 ± 1.18 | 8.59 ± 1.23 |
| Spleen | 9.03 ± 1.71 | 8.30 ± 0.87 | 9.66 ± 1.38 | 8.18 ± 1.74 | 7.89 ± 1.45 |
| Kidney | 9.56 ± 1.84 | 7.59 ± 0.24 | 9.32 ± 1.20 | 7.95 ± 1.11 | 5.92 ± 0.45 |
| Bladder | 4.99 ± 0.98 | 8.17 ± 1.23 | 9.18 ± 1.06 | 6.86 ± 1.06 | 6.84 ± 1.46 |
| Lung | 13.06 ± 2.52 | 10.23 ± 0.79 | 12.46 ± 1.15 | 8.97 ± 1.25 | 7.38 ± 0.97 |
| Gallbladder | 3.06 ± 1.29 | 2.49 ± 0.98 | 3.27 ± 1.82 | 3.30 ± 2.05 | 5.74 ± 2.83 |
| Muscle | 2.37 ± 0.50 | 4.27 ± 0.22 | 4.89 ± 0.50 | 3.20 ± 0.30 | 2.55 ± 0.21 |
| Fat | 3.19 ± 1.10 | 5.08 ± 1.15 | 3.92 ± 036 | 2.47 ± 0.86 | 2.35 ± 0.74 |
| Brain | 1.03 ± 0.44 | 0.77 ± 0.04 | 0.98 ± 0.31 | 0.74 ± 0.09 | 0.58 ± 0.15 |
| Uterus | 10.72 ± 8.98 | 10.50 ± 1.64 | 10.13 ± 1.78 | 9.07 ± 2.72 | 8.64 ± 3.90 |
| Ovaries | 6.00 ± 1.44 | 8.23 ± 0.72 | 8.02 ± 1.98 | 4.36 ± 1.13 | 4.53 ± 1.47 |
| Adrenals | 10.61 ± 2.57 | 9.04 ± 2.00 | 8.31 ± 3.74 | 6.49 ± 1.07 | 6.31 ± 1.67 |
| Thyroid | 6.82 ± 0.87 | 6.91 ± 2.06 | 7.70 ± 2.07 | 6.87 ± 1.51 | 4.69 ± 0.55 |
| Pancreas | 3.73 ± 0.96 | 5.21 ± 0.62 | 4.05 ± 0.52 | 3.20 ± 0.43 | 2.59 ± 0.43 |
| Thymus | 4.14 ± 1.59 | 4.12 ± 0..32 | 5.66 ± 1.68 | 3.97 ± 0.48 | 2.74 ± 0.56 |
| Stomach | 2.66 ± 0.60 | 2.34 ± 0.62 | 3.21 ± 0.62 | 2.13 ± 0.80 | 1.64 ± 0.79 |
| Sm Int | 4.21 ± 0.93 | 3.05 ± 0.47 | 3.73 ± 0.42 | 2.51 ± 0.50 | 1.89 ± 0.34 |
| U Lg Int | 4.02 ± 0.41 | 2.94 ± 0.27 | 4.12 ± 0.34 | 2.43 ± 0.55 | 1.71 ± 0.49 |
| L Lg Int | 2.81 ± 0.36 | 2.07 ± 0.09 | 2.55 ± 0.43 | 1.89 ± 0.32 | 1.37 ± 0.18 |

To estimate human dosimetry, integrated time-activity curves for [¹⁷⁷Lu]Lu-ofatumumab were calculated (TABLE 5). The longest (59.7 h) was in the blood, with extended time-activity curves seen in the blood-rich heart cavity, lung, and liver. Because of its large mass, muscle had the second longest time-activity curve, at 39 h. The adult human female model (Table 6) showed estimated dosimetry of 0.2-0.5 mSv/MBq in most organs, with the largest dose being to the heart wall (1.02 mSv/MBq) and lesser doses found for liver, spleen, and kidney (0.36, 0.48, and 0.43 mSv/MBq, respectively). Estimated doses to the osteogenic cells (bone surfaces) and red marrow were 0.82 and 0.54 mSv/MBq, respectively. The estimated effective dose was 0.36 mSv/MBq.

TABLE 5
Integrated time-activity for [177Lu]Lu-ofatumumab extrapolated
to humans. Analysis of C57BI/6N mice. Bladder content was estimated
using the excretion data and MIRD voiding bladder model assuming
3.2 h void interval. Sm Int, small intestine. U Lg Int, upper
large intestine. L L Int, lower large intestine.

| | Integrated time |
|---|---|
| Organ/Tissue | activity (h) |
| Blood | 59.70 |
| Lung | 5.57 |
| Liver | 5.31 |
| Spleen | 0.68 |
| Kidney | 1.30 |
| Bladder | 0.17 |
| Gallbladder | 0.02 |
| Muscle | 39.17 |
| Fat | 15.92 |
| Heart | 1.04 |
| Brain | 0.59 |
| Bone (surfaces) | 9.26 |
| Red marrow | 5.86 |
| Uterus | 0.47 |
| Ovaries | 0.04 |
| Adrenals | 0.05 |
| Thyroid | 0.16 |
| Pancreas | 0.25 |
| Thymus | 0.05 |
| Stomach | 0.19 |
| Small Int | 0.96 |
| U Lg Int | 0.13 |
| L Lg Int | 0.18 |
| Heart content | 5.37 |
| Bladder content | 0.31 |
| Excreted | 31.70 |
| Remainder of | 121.33 |
| body | |

TABLE 6
Human Radiation Dose Estimates for [¹⁷⁷Lu]Lu-
Ofatumumab Extrapolated to Adult Female Model

| Organ | mSv/MBq | rad/mCi |
|---|---|---|
| Adrenals | 0.39 | 1.44 |
| Brain | 0.05 | 0.19 |
| Breasts | 0.25 | 0.91 |
| Esophagus | 0.26 | 0.96 |
| Eyes | 0.25 | 0.91 |
| Gallbladder wall | 0.28 | 1.02 |
| Left colon | 0.36 | 1.34 |
| Small intestine | 0.41 | 1.50 |
| Stomach wall | 0.29 | 1.09 |
| Right colon | 0.29 | 1.08 |
| Rectum | 0.26 | 0.97 |
| Heart wall | 1.02 | 3.77 |
| Kidneys | 0.43 | 1.60 |
| Liver | 0.36 | 1.32 |
| Lungs | 0.53 | 1.96 |
| Ovaries | 0.39 | 1.44 |
| Pancreas | 0.21 | 0.77 |
| Salivary glands | 0.25 | 0.92 |
| Red marrow | 0.54 | 2.01 |
| Osteogenic cells | 0.82 | 3.02 |
| Spleen | 0.48 | 1.76 |
| Thymus | 0.26 | 0.96 |
| Thyroid | 0.80 | 2.97 |
| Urinary bladder wall | 0.34 | 1.27 |
| Uterus | 0.54 | 2.00 |
| Total body | 0.31 | 1.14 |
| Effective dose (mGy/MBq; | 0.36 | 1.34 |
| rem/mCi) | | |

Biodistribution of [¹⁷⁷Lu]Lu-Ofatumumab in Mice with Subcutaneous Raji-Cell Tumors

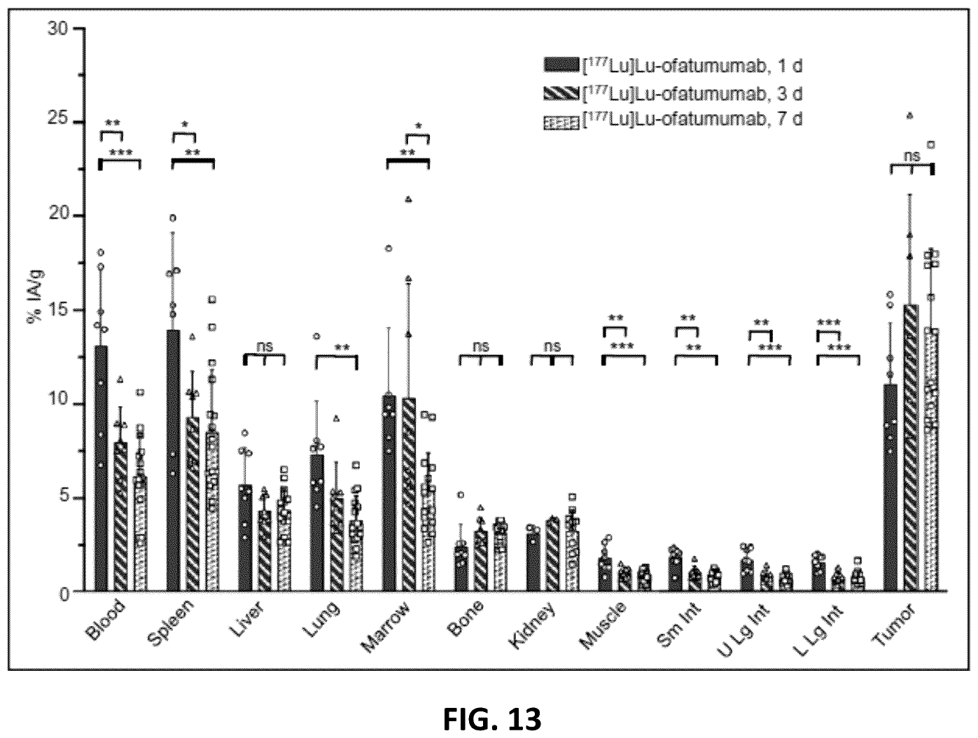

Biodistribution was investigated in R2G2 mice with subcutaneous Raji-cell tumors (FIG. 13). These mice are proficient in double-strand DNA-break repair and are less likely to show artifactual radiation toxicity than are repair-deficient Prkdc^{SCID} mice (16). [¹⁷⁷Lu]Lu-ofatumumab was injected at a low activity (370-444 kBq) to limit therapeutic effect, and biodistribution was determined 1, 3, and 7 d later (3-16 mice per time point). Blood decreased from about 13 to 6% IA/g, with a similar splenic distribution. Liver levels were about 5%, and marrow was 10% IA at 1 d and 5% IA/g at 7 d. Bone distribution was 2-3% IA/g. Tumor targeting was 11, 15, and 14% IA/g at 1, 3, and 7 d, respectively.

### Murine Therapy Study

To evaluate [¹⁷⁷Lu]Lu-ofatumumab therapeutic efficacy, R2G2 mice were injected intravenously with Raji-luc cells, and tumor cells were quantified by bioluminescent imaging (13). After injection, these cells disseminate to many organs (10,13,17,18), with hind-limb paralysis being a typical cause for killing of the animal due to growth in and around the spine.

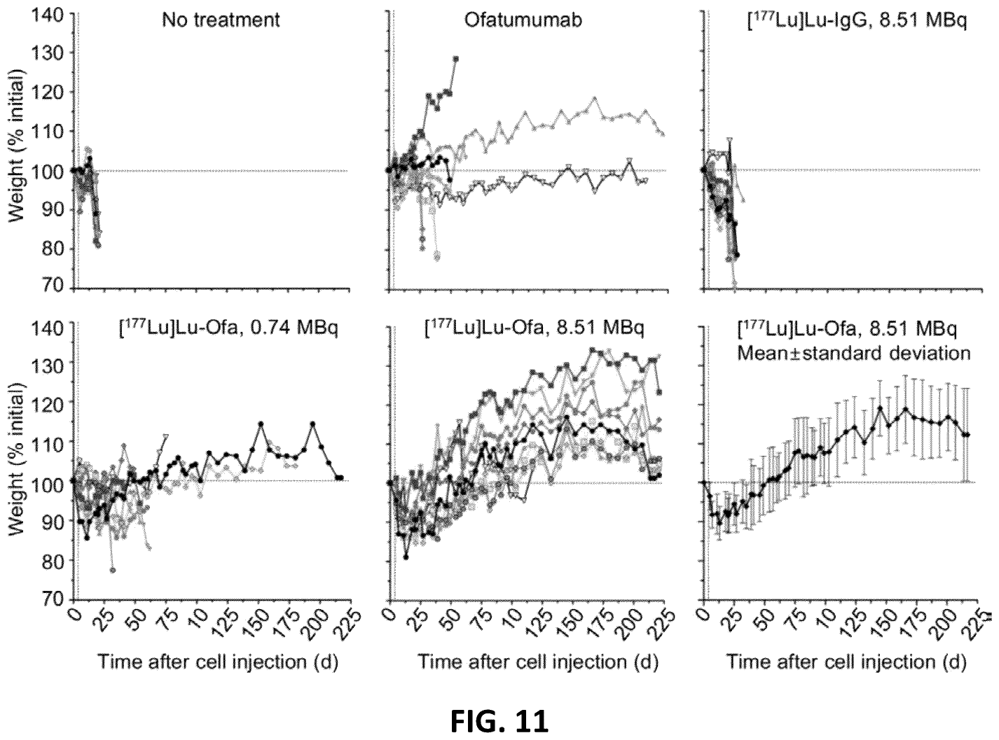

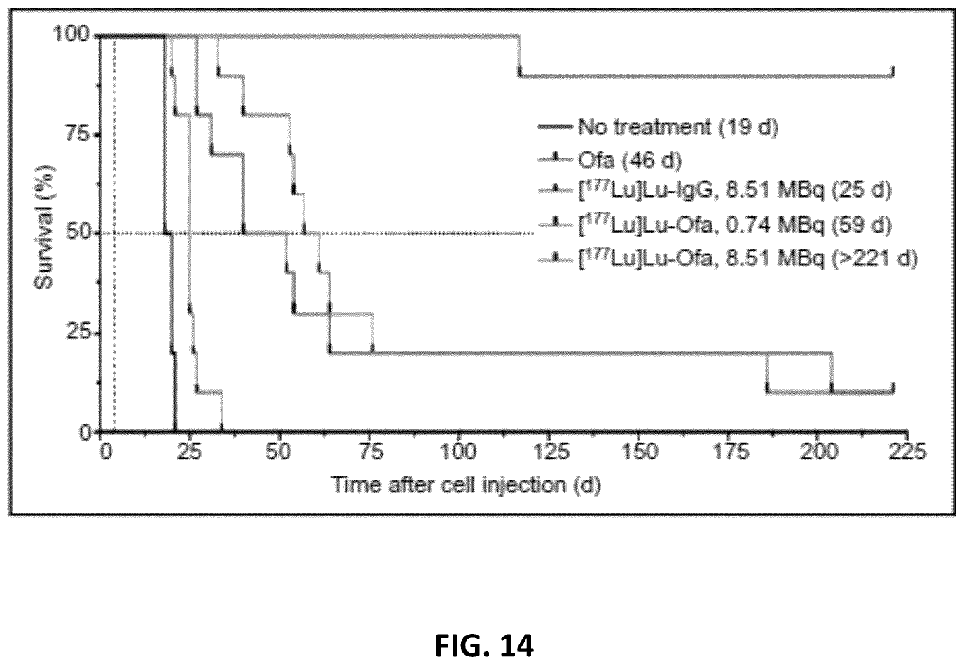

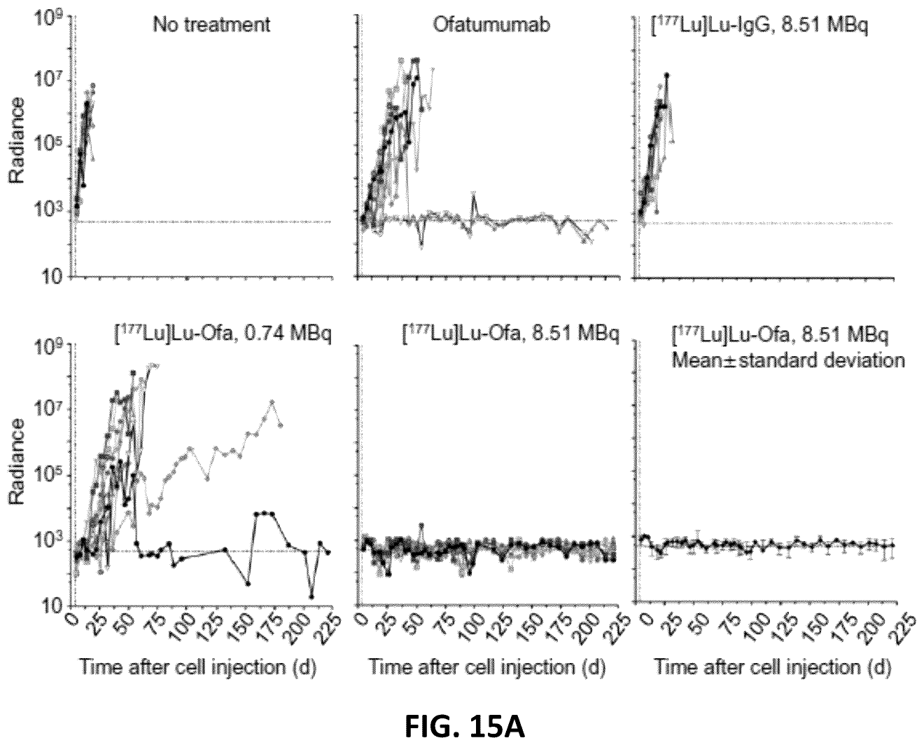

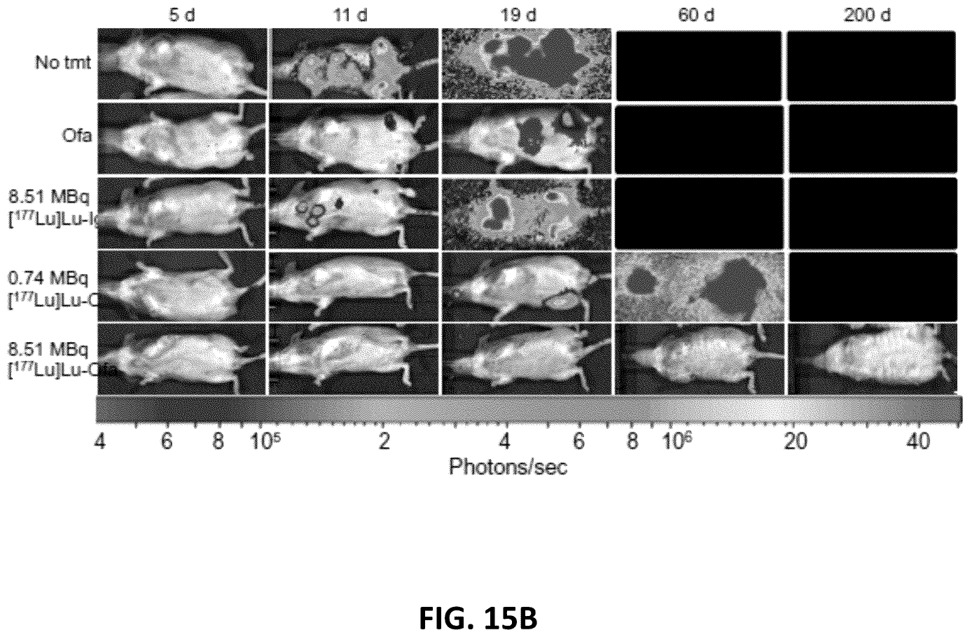

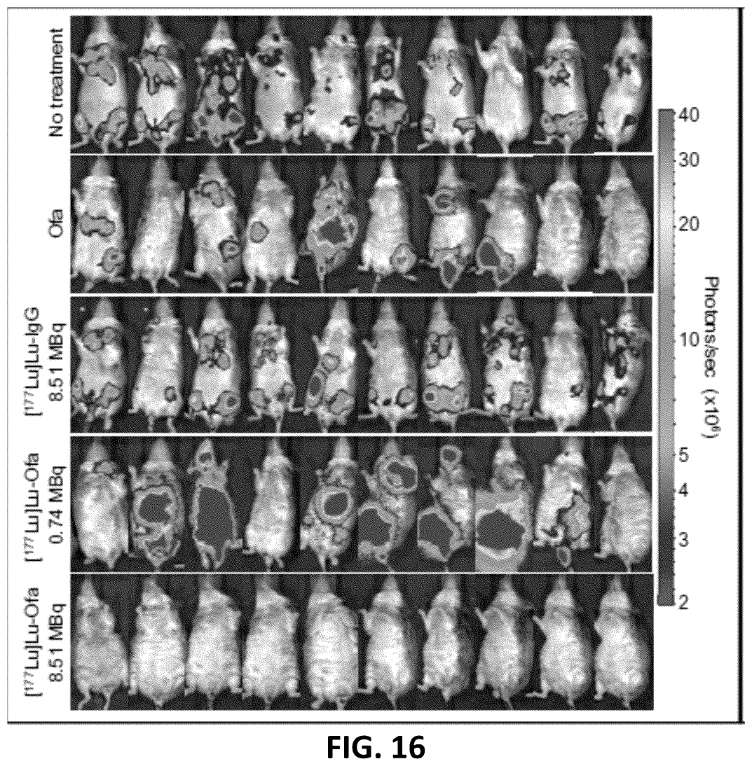

Four days after cell injection, the mice either were left untreated or were treated with native ofatumumab, 8.51 MBq of [¹⁷⁷Lu]Lu-human IgG1 (345±27 MBq/kg), 0.74 MBq (30±2.2 MBq/kg) of [¹⁷⁷Lu]Lu-ofatumumab, or 8.51 MBq (345±25.1 MBq/kg) of [¹⁷⁷Lu]Lu-ofatumumab (10 mice per group). Survival (FIG. 14), weight (FIG. 11), and bioluminescence (FIG. 15A) were tracked for 221 d. Representative bioluminescent images at selected time points are shown in FIG. 15B, and images of all mice just before they died or were killed for cause or study termination are shown in FIG. 16.

The median survival of untreated mice was 19 d, with none surviving beyond 22 d. Unlabeled ofatumumab yielded a median survival of 46 d, superior to untreated mice (Mantel-Cox, P<0.0001), with 1 mouse surviving without weight loss or increased bioluminescence. An 8.51-MBq dose of [¹⁷⁷Lu]Lu-IgG yielded 0 of 10 surviving mice and a median survival of 25 d, which was not different from that of untreated mice. For all 3 groups, increased bioluminescence and weight loss occurred before death or killing for cause.

A 0.74-MB dose of [¹⁷⁷Lu]Lu-ofatumumab yielded median survival of 59 d (9/10 mice not surviving), with increased bioluminescence and weight loss before death or killing for cause. This survival was superior to that of untreated mice (Mantel-Cox, P<0.0001) but not to that of mice receiving treatment with unlabeled ofatumumab. Hind-limb paralysis was frequently associated with death or killing for cause (TABLE 7).

TABLE 7
Phenotypes associated with death/sacrifice. Phenotypes associated
with death/sacrifice in therapy study of initiated 4 d post-
cell injection. n = 10 mice/group. HLP, hind-limb paralysis.
W, loss of >20% of initial weight. O, event other than
HLP or weight loss. Ofa, ofatumumab. Sur, survival.

| | HLP | W | O | Sur |
|---|---|---|---|---|
| No treatment | 10 | 0 | 0 | 0 |
| Ofa | 7 | 1 | 1 | 1 |
| [¹⁷⁷Lu]Lu-IgG, 8.51 MBq | 10 | 0 | 0 | 0 |
| [¹⁷⁷Lu]Lu-Ofa, 0.74 MBq | 7 | 1 | 1 | 1 |
| [¹⁷⁷Lu]Lu-Ofa, 8.51 MBq | 0 | 0 | 1 | 9 |

Notable therapeutic efficacy resulted from treatment with 8.51 MBq of [¹⁷⁷Lu]Lu-ofatumumab, with 9 of 10 mice surviving with continuous low bioluminescence (FIGS. 15 and 16). This survival was greater than that of untreated mice and of mice treated with unlabeled ofatumumab, 8.51 MBq of [¹⁷⁷Lu]Lu-IgG, or 0.74 MBq of [¹⁷⁷Lu]Lu-ofatumumab (Mantel-Cox, P<0.0003 for all comparisons). One mouse succumbed at 117 d, but this death appeared unrelated to tumor burden or therapy as no weight loss or increased bioluminescence occurred. Surviving mice displayed weight loss from 10 to 35 d after cell injection but recovered and gained weight.

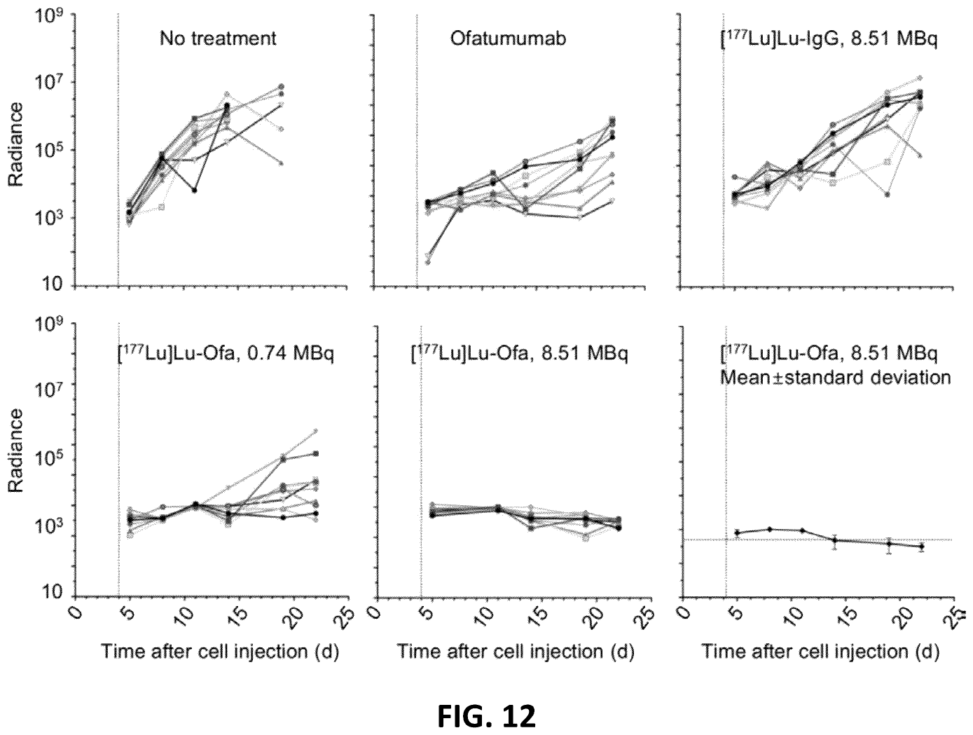

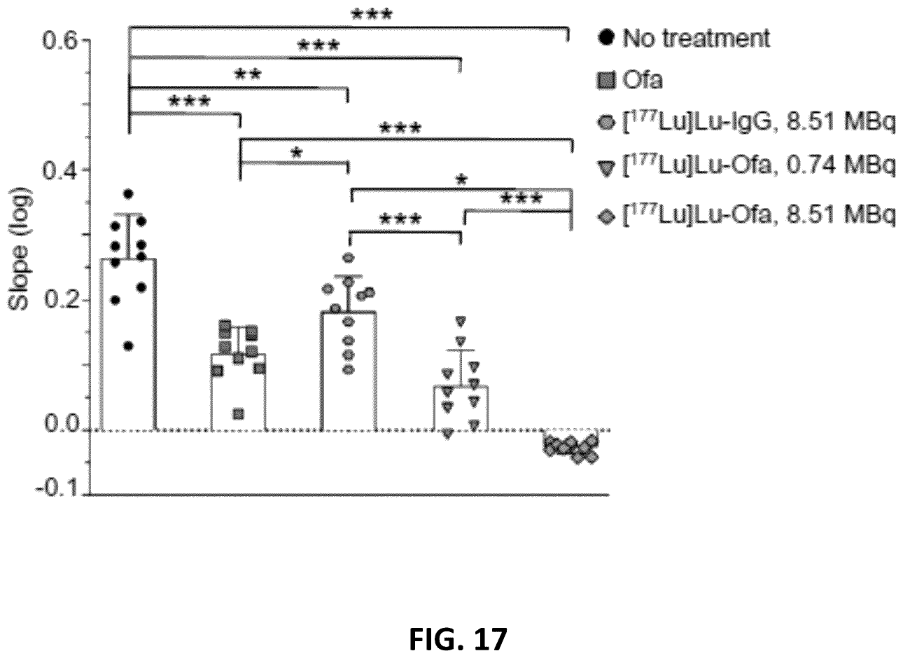

To determine how quickly therapy affected tumor cells, bioluminescence slopes from 1 to 18 d after initiation of therapy were compared (FIG. 17; FIG. 12). Compared with no treatment, ofatumumab, 8.51 MBq of [¹⁷⁷Lu]Lu-IgG, and 0.74 MBq of [¹⁷⁷Lu]Lu-ofatumumab slowed, but did not eliminate, tumor-cell proliferation. In contrast, 8.51 MBq of [¹⁷⁷Lu]Lu-ofatumumab quickly eliminated tumor cells, a finding that was significant compared with no treatment, treatment with unlabeled ofatumumab, treatment with 8.51 MBq of [¹⁷⁷Lu]Lu-IgG, or treatment with 0.75 MBq of [¹⁷⁷Lu]Lu-ofatumumab (P<0.05).

### Discussion

Our preclinical studies add to prior work demonstrating the potential of radiolabeled anti-CD20 antibodies to treat NHL. We show that [¹⁷⁷Lu]Lu-ofatumumab can be produced with high radiochemical yield and purity, excellent affinity, good stability and immunoreactivity, and potent cell killing. Additional advances include using a fully human anti-CD20 and ¹⁷⁷Lu, which have broad applicability in radiotherapy of cancer. In a model of rapidly progressing disease, we evaluated [¹⁷⁷Lu]Lu-ofatumumab therapy using dose-response studies and bioluminescence monitoring of tumor-cell burden. A single 8.51-MBq dose of [¹⁷⁷Lu]Lu-ofatumumab displayed curative efficacy.

Human dosimetry estimates predict that the highest dose from [¹⁷⁷Lu]Lu-ofatumumab (1.02 mSv/MBq) will be to the heart wall. The relatively radiation-resistant liver and spleen showed 0.36 and 0.48 mSv/MBq, respectively. The predicted dose to red marrow is 0.54 mSv/MBq, and hematologic toxicity likely will be dose limiting in clinical use, as was found with Bexxar, Zevalin, [¹⁷⁷Lu]Lu-J591 (19), [¹⁷⁷Lu]Lu-G250 anti-CAIX (20), and [¹⁷⁷Lu]Lu-rituximab (21). As 2 Sv is a typical maximal dose for acceptable hematologic toxicity without stem cell support, delivering this radiation to the marrow would be tolerable. As there may be patient-to-patient variability with [¹⁷⁷Lu]Lu-ofatumumab due to cross reactivity with normal CD20-positive cells, our dosimetry data provide guidance for activity administration to humans. Dosimetric estimation could also potentially be obtained using a PET imaging surrogate, such as [⁸⁹Zr]Zr-ofatumumab (9,22).

The stable in vivo chelation of ¹⁷⁷Lu by CHX-A″-DTPA-ofatumumab agrees with the results of others using this chelator-radionuclide combination (23,24). Although it has been suggested that, for stable ¹⁷⁷Lu chelation, macrocyclic DOTA requires high temperatures incompatible with maintaining antibody function (24,25), experiments show that this is not the case (26,27). Thus, CHX-A″-DTPA and DOTA both appear practical for chelation of ¹⁷⁷Lu to antibodies and antibody fragments.

Others have used [¹⁷⁷Lu]Lu-anti-CD20 intact antibodies or ¹⁷⁷Lu-labeled antibody-based radiopharmaceuticals for preclinical and clinical therapy. Ertveld et al. (23), using a single-domain anti-CD20 antibody in immunocompetent mice with CD20-expressing subcutaneous tumors, found a modest therapeutic effect at 140 MBq/mouse; 50 MBq/mouse induced expression of proinflammatory genes, whereas 140 MBq/mouse increased the percentage in the tumor of PD-L1-positive myeloid cells and alternatively activated macrophages. Krasniqi et al. (28) compared a single-domain anti-CD20 antibody with unlabeled rituximab and [¹⁷⁷Lu]Lu-CHX-A″-DTPA-rituximab in mice with CD20-expressing subcutaneous tumors. All treatments increased survival over no treatment, but [¹⁷⁷Lu]Lu-CHX-A″-DTPA-rituximab was only slightly better than rituximab. In a phase I/II study of [¹⁷⁷Lu]Lu-DOTA-rituximab in 31 patients with relapsed or refractory CD20-positive lymphoma, mainly hematologic toxicity was observed, with frequent tumor responses and 8 of 11 patients with follicular lymphoma alive after an 84-mo median follow-up (29).

A major finding of the current study is the high therapeutic efficacy of [¹⁷⁷Lu]Lu-ofatumumab in a murine model of disseminated lymphoma. Therapy was initiated 4 d after intravenous cell injection, when tumor cells are present individually or as small groups, comparable to micrometastatic or minimal residual disease in humans. An 8.51-MBq dose of [¹⁷⁷Lu]Lu-ofatumumab reduced tumor burden within about 2 d and eliminated bioluminescence-detectable tumors, with 9 of 10 mice still alive 221 d later. This response was dose-dependent and specific, as 0.74 MBq of [¹⁷⁷Lu]-Lu ofatumumab and 8.51 MBq of [¹⁷⁷Lu]-IgG did not extend survival or prevent tumor-cell proliferation. Although attenuation from tissue, skin, and fur means that bioluminescent imaging may not detect a low tumor-cell burden (13), the durability of the response suggests complete elimination of tumor cells by 8.51 MBq of [¹⁷⁷Lu]Lu-ofatumumab. After initial weight loss, these mice gained weight, suggesting no or low whole-body toxicity. The internalization of ofatumumab after CD20 binding (30) and the residualization of ¹⁷⁷Lu within the cell may contribute to its therapeutic efficacy. Moreover, the lack of murine sequences in [¹⁷⁷Lu]Lu-ofatumumab suggests a potential for fractionated therapy or repeated treatments. In an interesting approach, with relatively small subcutaneous tumors of rituximab-resistant Raji cells, Malenge et al. (26) combined [¹⁷⁷Lu]Lu-lilotomab (anti-CD37) and unlabeled rituximab, with good therapeutic results.

α-particle therapy is another potential approach to treating lymphoma. Using a murine Raji-cell disseminated lymphoma model, [²¹³Bi]Bi-rituximab (t_{1/2}, 45.6 min) was typically curative when tumor burden was low (4 d after cell injection) but not when it was higher (18), perhaps because of lack of time to target larger tumor masses before decay. Similarly, [¹⁴⁹Tb]Tb-rituximab (t_{1/2}, 4.2 h) therapy initiated 2 d after Daudi-cell intravenous injection increased survival (31). A 1F5 anti-CD20 antibody with chelated ²¹¹At (t_{1/2}, 7.2 h) was 80% curative when injected 6 d after intravenous cell injection with supporting stem-cell transplantation but only slowly reduced the growth of subcutaneous tumors (32). On the basis of these results and on the multiday tumor-targeting pharmacokinetics of intact antibodies, radioimmunotherapy of larger tumor masses with intact antibodies will likely be most successful using radioisotopes that permit tumor localization before decay, including ¹⁷⁷Lu or α-particle-emitting ²²⁵Ac with its 10-d half-life.

Our studies add to the literature demonstrating the effectiveness of ¹⁷⁷Lu-radiopharmaceuticals in cancer therapy. We found remarkable effectiveness in micrometastatic disease, and the 6.6-d half-life and multiple-cell-diameter killing range of ¹⁷⁷Lu suggests that [¹⁷⁷Lu]Lu-ofatumumab may be effective against larger tumors.

Although initial anti-CD20 radioimmunotherapies showed limited commercial success for several reasons, we suggest that a reevaluation of next-generation β- and α-particle therapies is in order. [¹⁷⁷Lu]Lu-ofatumumab CD20-targeted radioimmunotherapy may be an effective approach for therapy of NHL or other CD20-expressing diseases.

### Conclusion

Chx-A″-DTPA-ofatumumab stably chelates 177Lu in vitro and in vivo, and [¹⁷⁷Lu]Lu-Chx-A″-ofatumumab effectively targets CD20-expressing tumor xenografts. In a mouse model of disseminated human lymphoma, therapy with [¹⁷⁷Lu]Lu-ofatumumab showed curative therapeutic efficacy.

What is claimed is:

1. A method of treating disseminated human lymphoma in a subject in need thereof, the method comprising applying a theranostic treatment comprising at least one of [²²⁵Ac]Ac-Ofatumumab and [177Lu]Lu-ofatumumab.

2. A method of treating disseminated human lymphoma in a subject in need thereof, the method comprising applying a theranostic treatment comprising a combination of [²²⁵Ac]Ac-Ofatumumab and [89Zr]Zr-Ofatumumab.

3. A method of treating a CD20 disease in a subject in need thereof, the method comprising applying a theranostic treatment comprising at least one of [²²⁵Ac]Ac-Ofatumumab, [177Lu]Lu-ofatumumab, and a combination of [²²⁵Ac]Ac-Ofatumumab and [89Zr]Zr-Ofatumumab.
